(19) United States
(12) Patent Application Publication
(10) Pub. No.: US 2013/0197088 A1
(43) Pub. Date: Aug. 1, 2013

(54) Compositions and Methods for Combinations of Oligoamines with 2-Difluoromethylornithine (DFMO)

(71) Applicants: Robert A. Casero, JR., Glen Arm, MD (US); Laurence J. Marton, Palo Alto, CA (US)

(72) Inventors: Robert A. Casero, JR., Glen Arm, MD (US); Laurence J. Marton, Palo Alto, CA (US)

(21) Appl. No.: 13/634,237

(22) PCT Filed: Mar. 11, 2011
(86) PCT No.: PCT/US11/28206

§ 371 Date: Nov. 14, 2012

Related U.S. Application Data
(60) Provisional application No. 61/313,313, filed on Mar. 12, 2010.

(51) Int. Cl. A61K 31/198; A61K 31/132; A61K 31/155

(52) U.S. Cl. CPC A61K 31/198; A61K 31/155; A61K 31/132
USPC 514/564; 435/375

(57) ABSTRACT

The present invention is based on the seminal discovery of a synergistic effect for combinations of oligoamines with 2-difluoromethylornithine (DFMO) for treatment of cancer. The invention provides combinations of at least one inhibitor of a histone demethylase enzyme and at least one inhibitor of ornithine decarboxylase (ODC). The invention also provides methods for treatment of cancer using such combinations and methods for altering methylation in a cell using such combinations. The invention provides that certain silenced genes can be re-expressed using combinations disclosed herein.

### BACKGROUND OF THE INVENTION

1. Field of the Invention

The invention relates generally to combination therapy for treatment of cancer, and more specifically to compositions and methods of use a combination of oligoamines with 2-difluoromethylornithine (DFMO).

2. Background Information

Epigenetic gene silencing is an important mechanism for the loss of gene expression in cancer. Abnormal DNA CpG island hypermethylation and altered patterns of histone modifications are involved in aberrant silencing of tumor suppressor gene. The flavin-dependent lysine-specific demethylase 1 (LSD1) is the first enzyme identified to specifically demethylate Lysine 4 of histone H3. Methylation of H3K4 is an important mark associated with active chromatin transcription. The FAD-binding amine oxidase domain of LSD1 has considerable sequence homology to two polyamine oxidases SMO (spermine oxidase) and APAO (acetylpolyamine oxidase).

Epigenetic changes in chromatin including methylation of gene promoter region CpG islands collaborate with histone modifications, including histone methylation, acetylation, and phosphorylation to regulate gene expression. Aberrant epigenetic silencing of gene expression plays a key role in the genesis and progression of cancer. Specific polyamine analogues have been demonstrated to inhibit the chromatin-remodeling and transcriptional repressive enzyme, lysine specific demethylase 1 (LSD1). In specific instances, inhibition of LSD1 by polyamine analogues results in the re-expression of genes that are aberrantly silenced in cancer. These results have been confirmed both in vitro and in vivo. In vivo treatment of established human tumors in nude mice demonstrated that long chain polyamine analogues (oligoamines) effectively inhibited LSD1 in situ and resulted in a dramatic decrease in tumor size. In vitro results with the oligoamines demonstrated that treatment of various different cancer cell types including colon, breast, and leukemias resulted in increased methylated lysine 4 of histone 3, the target of LSD1 and increased expression of various previously silenced genes. Each of these results is consistent with the hypothesis that inhibition of LSD1 by the oligoamines is responsible for the re-expression of the silenced genes.

### SUMMARY OF THE INVENTION

The present invention is based on the seminal discovery of a synergistic effect for a combination of oligoamines with 2-difluoromethylornithine (DFMO) for the treatment of cancer. In one aspect, the invention provides combinations of at least one inhibitor of a histone demethylase enzyme and at least one inhibitor of ornithine decarboxylase (ODC). The invention also provides methods for treatment of cancer using such combinations and methods for altering methylation in a cell using such combinations. The invention provides that certain silenced genes can be re-expressed using combinations disclosed herein.

In one embodiment, the present invention provides a composition including (a) a therapeutically effective amount of at least one inhibitor of a histone demethylase enzyme; and (b) a therapeutically effective amount of at least one inhibitor of ornithine decarboxylase (ODC).

In one aspect, the histone demethylase enzyme includes lysine-specific demethylase 1 (LSD1). In another aspect, the inhibitor of LSD1 includes a polyamine. In another aspect, the composition is with the proviso that the inhibitor of a histone demethylase enzyme does not include a natural polyamine.

In one aspect, the histone demethylase enzyme includes Jumonjii domain-containing (JmjC) histone demethylase. In an additional aspect, the JmjC histone demethylase is PHF8 or KIAA1718. In another aspect, the inhibitor of ODC includes 2-difluoromethylornithine (DFMO or alpha-difluoromethylornithine). In an additional aspect, the inhibitor of ODC includes enriched D-enantiomer of DFMO.

In one aspect, the polyamine of the disclosed composition includes a compound of formula (I):

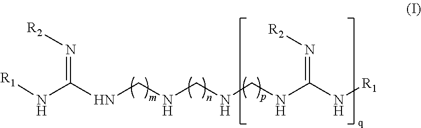

or a pharmaceutically acceptable salt thereof, wherein:
n is an integer from 1 to 12;
m and p are each independently an integer from 1 to 5;
q is 0 or 1;
each R₁ is independently selected from the group consisting of:
C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, and C₇-C₂₄ substituted or unsubstituted heteroaralkyl and;
each R₂ is independently selected from hydrogen or a C₁-C₈ substituted or unsubstituted alkyl.

In another aspect, the polyamine of the disclosed composition includes an oligoamine of formula (X):

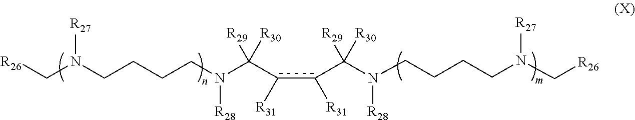

or a pharmaceutically acceptable salt thereof, wherein:
n and m are each independently an integer from 1 to 12;
each R₂₆, R₂₇, R₂₈, R₂₉, R₃₀, and R₃₁ is independently selected from hydrogen, a C₁-C₈ substituted or unsubstituted alkyl, a C₆-C₂₀ substituted or unsubstituted aryl, and an amine; and [symbol] is a single bond or double bond.

In another aspect, the compound of the disclosed composition is selected from

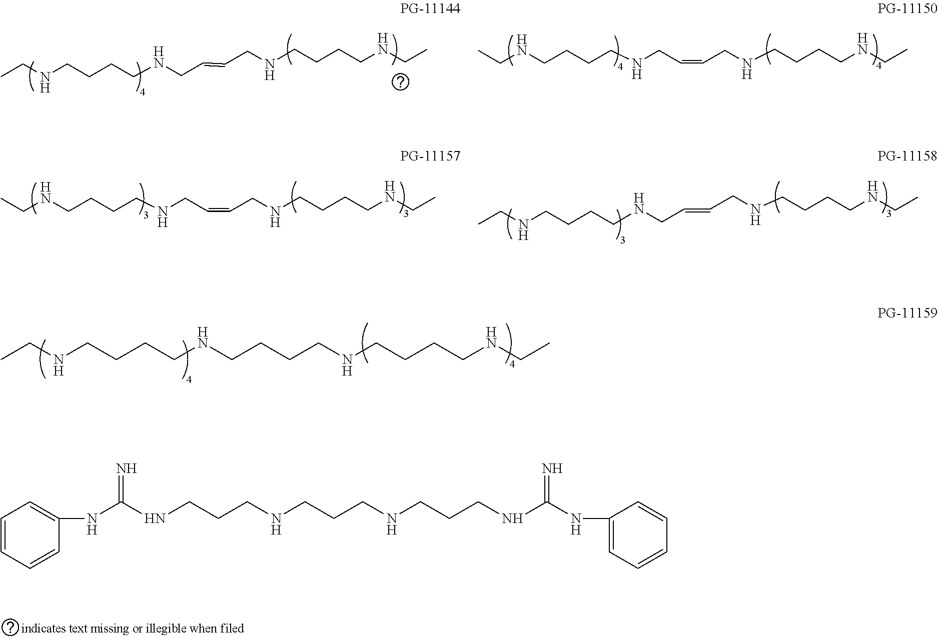

and a combination thereof.

In various aspects, the dosage for DFMO used is less than about 100 mg/kg. In various aspects, the dosage for DFMO used is less than about 10 mg/kg. In various aspects, the dosage for DFMO used is less than about 1 mg/kg. In various aspects, the dosage for DFMO used is less than about 0.1 mg/kg. In various aspects, the dosage for DFMO used is between about 0.1-10 mg/kg. In various aspects, the dosage for the polyamine of the invention is less than about 100 μg/kg. In various aspects, the dosage for the polyamine of the invention is less than about 10 μg/kg. In various aspects, the dosage for the polyamine of the invention is less than about 1 μg/kg. In various aspects, the dosage for the polyamine of the invention is less than about 0.1 μg/kg. In various aspects, the dosage for the polyamine of the invention is between about 0.1-10 μg/kg.

In another embodiment, the present invention provides a method for treatment of cancer in a subject. The method includes administering to the subject a therapeutically effective amount of at least one inhibitor of a histone demethylase enzyme in combination with a therapeutically effective amount of at least one inhibitor of ornithine decarboxylase (ODC).

In one aspect, the inhibitor of ODC includes 2-difluoromethylornithine (DFMO or alpha-difluoromethylornithine) and the inhibitor of a histone demethylase enzyme includes a polyamine. In another aspect, the method is with the proviso that the inhibitor of a histone demethylase enzyme does not include a natural polyamine.

In various aspects, the polyamine of the method disclosed includes a compound of formula (I) or formula (X):

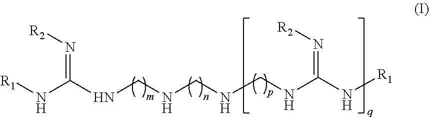

or a pharmaceutically acceptable salt thereof, wherein:
n is an integer from 1 to 12;
m and p are each independently an integer from 1 to 5;
q is 0 or 1;
each R₁ is independently selected from the group consisting of:
C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, and C₇-C₂₄ substituted or unsubstituted heteroaralkyl and;
each R₂ is independently selected from hydrogen or a C₁-C₈ substituted or unsubstituted alkyl; or

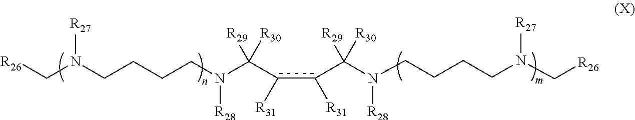

or a pharmaceutically acceptable salt thereof, wherein:
n and m are each independently an integer from 1 to 12;
each R₂₆, R₂₇, R₂₈, R₂₉, R₃₀, and R₃₁ is independently selected from hydrogen, a C₁-C₈ substituted or unsubstituted alkyl, a C₆-C₂₀ substituted or unsubstituted aryl, and an amine; and [symbol] is a single bond or double bond.

In various aspects, the compound of the method disclosed is selected from

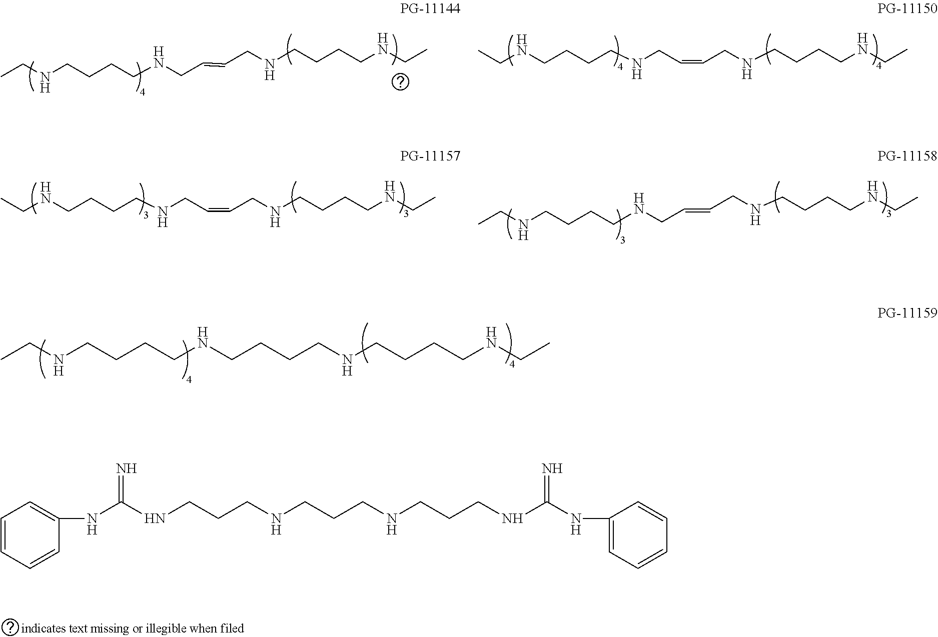

and a combination thereof. In various aspects, the subject is a human patient.

In another embodiment, the present invention provides a method of altering DNA methylation in a cell. The method includes administering the cell with at least one inhibitor of a histone demethylase enzyme in combination with at least one inhibitor of ornithine decarboxylase (ODC).

In one aspect, the inhibitor of ODC includes 2-difluoromethylornithine (DFMO or alpha-difluoromethylornithine) and the inhibitor of a histone demethylase enzyme includes a polyamine. In another aspect, the method is with the proviso that the inhibitor of a histone demethylase enzyme does not include a natural polyamine. In another aspect, the polyamine of the method disclosed includes a compound of formula (I) or formula (X) as described above. In another aspect, the compound of the method disclosed is selected from specific compounds described above.

In various aspects, the cancer is selected from the group consisting of bladder, brain, breast, colon, esophagus, kidney, liver, lung, mouth, ovary, pancreas, prostate, skin, stomach, hematopoietic system and uterus. In various aspects, the hematopoietic cancers include at least one of acute myeloid leukemia, mesothelioma, cutaneous T-cell lymphoma (CTCL), multiple myeloma and myelodysplastic syndrome (refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) or combinations thereof.

In another embodiment, the present invention provides a method for enhancing inhibition of a histone demethylase enzyme in a cell. The method includes (a) administering the cell with at least one inhibitor of ornithine decarboxylase (ODC) and (b) administering the cell with at least one inhibitor of a histone demethylase enzyme.

In one aspect, the inhibitor of ODC includes 2-difluoromethylornithine (DFMO or alpha-difluoromethylornithine) and the inhibitor of a histone demethylase enzyme includes a polyamine. In another aspect, the method is with the proviso that the inhibitor of a histone demethylase enzyme does not include a natural polyamine. In another aspect, the step (a) includes a pretreatment period from about 2 hours to about 48 hours. In another aspect, the step (a) includes a pretreatment period from about one day to about one week. In another aspect, the step (a) includes a pretreatment period of at least 10 hours. In another aspect, the step (a) includes a pretreatment period of at least 24 hours.

In another aspect, the polyamine of the method disclosed includes a compound of formula (I) or formula (X) as described above. In another aspect, the compound of the method disclosed is selected from specific compounds described above. In various aspects, the subject is human. In various aspects, the cell includes a cancer cell. In various aspects, the cell includes a colorectal cancer cell. In various aspects, the compositions and/or methods disclosed are useful for treatment of cancer. In various aspects, the cancer is colorectal cancer. In other aspects the cancer is bladder, brain, breast, colon, esophagus, kidney, liver, lung, mouth, ovary, pancreas, prostate, skin, stomach, hematopoietic system or uterus. In various aspects, the cell to which the inhibitors of the invention are administered may be performed in vivo (for example an individual cell or a cell that is part of a tissue or an organ within a subject), ex vivo (for example in cell cultures), or a combination thereof. In various aspects, the methods disclosed are useful for diagnostic purpose, treatment of diseases, or a combination thereof.

In another embodiment, the present invention provides the use of at least one inhibitor of a histone demethylase enzyme in combination with at least one inhibitor of ornithine decarbosylase (ODC) in the manufacture of a medicament for treating cancer in a subject. In another embodiment, the present invention provides a combination of at least one inhibitor of a histone demethylase enzyme and at least one inhibitor of ornithine decarbosylase (ODC) for use in a method of treating cancer in a subject.

In one aspect, the inhibitor of ODC includes 2-difluoromethylornithine (DFMO or alpha-difluoromethylornithine) and the inhibitor of a histone demethylase enzyme includes a polyamine. In another aspect, the use or combination provided is with the proviso that the inhibitor of a histone demethylase enzyme does not include a natural polyamine.

In various aspects, the polyamine of the use or combination provided includes a compound of formula (I) or formula (X):

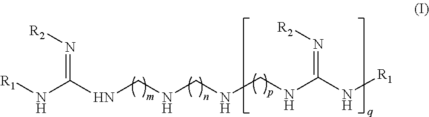

or a pharmaceutically acceptable salt thereof, wherein:
n is an integer from 1 to 12;
m and p are each independently an integer from 1 to 5;
q is 0 or 1;
each R₁ is independently selected from the group consisting of:
C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, and C₇-C₂₄ substituted or unsubstituted heteroaralkyl and;
each R₂ is independently selected from hydrogen or a C₁-C₈ substituted or unsubstituted alkyl; or

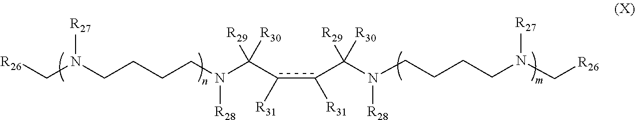

or a pharmaceutically acceptable salt thereof, wherein:
n and m are each independently an integer from 1 to 12;
each R₂₆, R₂₇, R₂₈, R₂₉, R₃₀, and R₃₁ is independently selected from hydrogen, a C₁-C₈ substituted or unsubstituted alkyl, a C₆-C₂₀ substituted or unsubstituted aryl, and an amine; and [symbol] is a single bond or double bond.

In various aspects, the compound of the use or combination provided is selected from

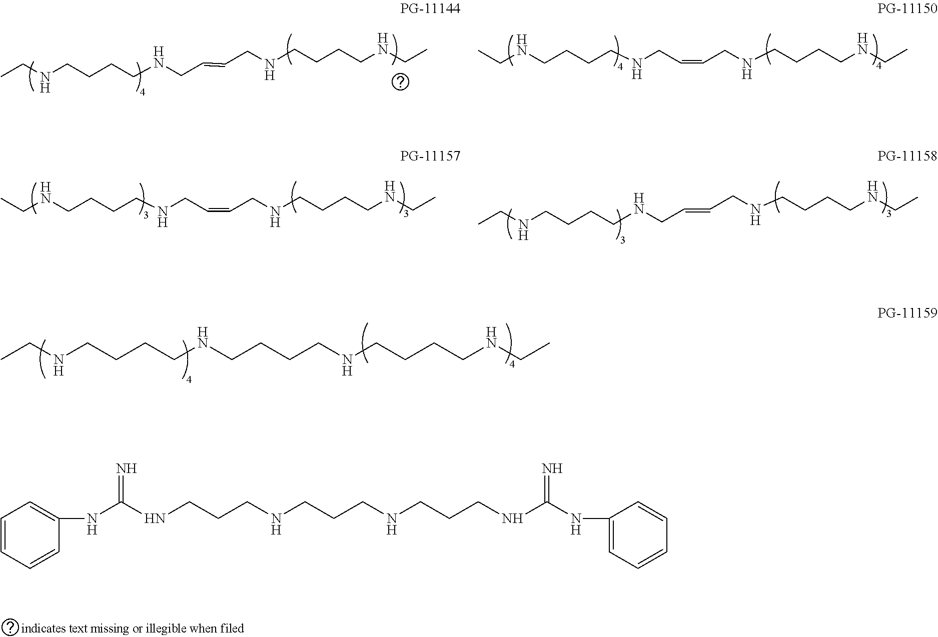

and a combination thereof. In various aspects, the subject is a human patient.

### BRIEF DESCRIPTION OF THE DRAWINGS

FIG. 1 shows that combination of DFMO with oligoamines produces a synergistic increases global H3K4me2. FIG. 1A shows exemplary chemical structures of the oligoamines disclosed herein. FIGS. 1B, 1C, and 1D show that HCT116 cells are first treated for 24 hours with 5 mM DFMO followed by another 24 hour treatment of replenished 5 mM DFMO and oligoamines (PG-11144, PG-11150 and PG-11157 for panel B, PG 11158 and PG-11159 for panel C) alone or simultaneously. Nuclear fractions are prepared using NE-PER Nuclear and Cytoplasmic Extraction reagents. 50 μg of nuclear protein/lane are analyzed by Western blotting analysis for expression of H3K4me2, PCNA is shown as a loading control. Shown are representative Western blotting images of triplicate treatments. Relative protein expression levels were determined by quantitative Western analysis using the Odyssey infrared detection system shown as bar graphs. The results represent the mean of three treatments±standard deviation (SD or S.D.). The protein expression level for control samples is set to a value of 1.

FIG. 2 shows synergy of oligoamines and DFMO in the re-expression of aberrantly silenced SFRP2. HCT116 cells are first treated for 24 hour with 5 mM DFMO followed by another 24 hour treatment of replenished 5 mM DFMO and oligoamines (PG-11144 and PG-11150 for panel A, PG-11157, PG 11158 and PG-11159 for panel B) alone or simultaneously. RNA is extracted using TRIzol reagents and first-strand cDNA is synthesized using M-MLV reverse transcriptase with an oligo(dT) primer (Invitrogen). qPCR for SFRP2 is performed in a MyiQ single color real-time PCR machine with GAPDH as an internal control. The SFRP2 primers used for qPCR are: sense, 5′ AAG CCT GCA AAA ATA AAA ATG ATG (SEQ ID NO: 1); antisense, 5′ TGT AAA TGG TCT TGC TCT TGG TCT (SEQ ID NO: 2) (annealing at 53° C.). The quantified results are the mean of triplicate treatments. The transcript level for control samples was set to a value of 1. S.D. is indicated by the error bars.

FIG. 3 shows dose response of PG-11144 with co-treatment of DFMO in the synergistic re-expression of SFRP2. HCT116 cells are first treated for 24 hours with 5 mM DFMO followed by another 24 hours treatment of replenished 5 mM DFMO and PG-11144 in the indicated doses alone or simultaneously. RNA isolation and qPCR are performed. The quantified results are the means of triplicate treatments with S.D. as indicated. The transcript level for control samples is set to a value of 1.

FIG. 4 shows an exemplary pathway to synthesize various polyamine analogs.

FIG. 5 shows that DFMO in combination of PG-11144 synergistically increases activating H3K4me2 mark at the promoters of SFRP2. HCT116 cells are first treated for 24 hours with 5 mM DFMO followed by another 24 hours treatment of replenished 5 mM DFMO and 2.5 μM PG-11144 alone or simultaneously. Chromatin immunoprecipitation (CHIP) analysis is performed using EZ-chip kit (Millipore). In brief, cells are exposed to 1% formaldehyde to cross-link proteins, and two million cells are used for each CHIP assay. Antibodies against H3K4me2, and for control, H3 are used as indicated for immunoprecipitation of protein-DNA complexes. Quantitative ChIP is performed using qPCR on the MyiQ single color real-time PCR machine. The PCR primer sets used for amplification of precipitated SFRP2 promoter fragments are as follows: sense, 5′ CTC CCT CCC AGC CTG CCC ATC TT (SEQ ID NO: 3); antisense, 5′ ACT GCC CAC CAT TTC CCC GTT TTG (SEQ ID NO: 4) (annealing at 61° C.). The relative enrichment of H3K4me2 for control samples is set to a value of 1.

### DETAILED DESCRIPTION OF THE INVENTION

The present invention provides that certain long chain polyamine analogues, named oligoamines, act as inhibitors of lysine-specific demethylase 1 (LSD1). The present invention provides unexpected synergistic effects of specific oligoamines in combination treatment with 2-difluoromethylornithine (DFMO), especially in human colorectal cancer cells. DFMO is an inhibitor of ornithine decarboxylase (ODC), which is a rate-limiting enzyme to generate putrescine. Reducing the level of putrescine leads to accumulation of decarboxylated S-adenosylmethionine (dcSAM) and can subsequently result in alteration of DNA methyltransferase activity and changes in DNA methylation. In addition, DFMO treatment is known to increase uptake of circulating polyamines. Exposure of colorectal tumor cells to oligoamines and DFMO results in a synergistically global increase of H3K4me2 and induction of re-expression of aberrantly silenced genes including the secreted frizzled-related protein 2 (SFRP2) gene, which encodes Wnt signaling pathway antagonist and plays an anti-tumorigenesis role in colorectal cancer. Chromatin immunoprecipitation analysis indicates that the re-expression of SFRP2 is associated with increased H3K4me2 active marks at the gene promoter. The combination of LSD1-inhibiting oligoamines and DFMO represents a unique, highly valuable, and novel approach to epigenetic therapy of cancer.

The natural polyamines disclosed include polyamine cationic alkylamines that positively charged at physiologic pH. They are closely associated with chromatin and are thought to have a role in the regulation of multiple cellular functions, including gene expression. An inhibitor of the first, rate-limiting step in polyamine biosynthesis, ornithine decarboxylase, 2-difluoromethylornithine (DFMO) can be used to greatly reduce intracellular polyamine concentrations, both in vitro and in vivo. The present invention provides that the reduction of the natural polyamines in cancer cells by pretreatment with DFMO can enhance the epigenetic effects of oligoamine treatment through two mechanisms. 1) The reduction of the natural polyamines would allow the analogues to have greater access to their targets and; 2) the reduction of natural polyamines is known to result in increased uptake of polyamine like compounds, thus possibly increasing the effective intracellular concentrations of the oligoamines. Therefore, the present invention provides effects of combination treatment of human tumor cells with DFMO and the oligoamine analogues. As the requirement for, and the metabolism of, polyamines are frequently dysregulated in cancer, this combination of agents could also be expected to be relatively selective for tumor cells, thus potentially increasing the therapeutic index of the combination.

The present invention provides that the combination of the polyamine depleting treatment with DFMO can lead to increased expression of tumor suppressor genes in human colon cancer cells that exceeds that induced by LSD1 inhibition alone. The surprising results disclosed here indicate that not only does this combination increase the effectiveness it actually produces synergistic effects both with regards to inhibition of LSD1 activity and increased expression of aberrantly silenced tumor suppressor genes.

A “therapeutically effective amount” or “pharmaceutically active amount” refers to an amount at least partially effective, at dosages and for periods of time necessary, to achieve the desired therapeutic result to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. A “therapeutically effective amount” or “pharmaceutically active amount” is an amount sufficient to at least partially affect beneficial or desired results, including clinical results. An effective amount can be administered in one or more administration. The administration can be sequential or simultaneous for example. When simultaneous, the administration can be a co-administration in a single dosage format or separately administered. For purposes of this invention, an effective amount of an adenoviral vector is an amount that is sufficient to at least partially palliate, ameliorate, stabilize, reverse, slow or delay the progression of the disease state.

In another embodiment, the active agent according to the methods of the invention is formulated in the composition in a prophylactically effective amount. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, the phrase “polyamine” refers to generally a molecule having more than one amine group. The phrase “oilogoamine” refers to a molecule having repeated units (as monomers) and each unit has at least one amine groups. Thus, in some embodiments, an oligoamine of the invention can be a polymer having monomer units of polyamines. In some embodiments, an oligoamine is also a polyamine because the oligoamine has more than one amine group. In some embodiments, the oligoamines or polyamines of the invention exclude natural polyamines. Such natural polyamines include, for example, putresine, spermidine, or spermine.

As used herein, “treatment” of a subject includes the application or administration of a therapeutic agent to a subject, or application or administration of a therapeutic agent to a cell or tissue from a subject, who has a diseases or disorder (e.g., cancer), has a symptom of a disease or disorder, or is at risk of (or susceptible to) a disease or disorder, with the purpose of curing, healing, alleviating, relieving, altering, remedying, ameliorating, improving, or affecting the disease or disorder, the symptom of the disease or disorder, or the risk of (or susceptibility to) the disease or disorder.

“Treating” or “to treat” a disease using the methods of the invention is defined as administering one or more polyamines or polyamine analogs, with or without additional therapeutic agents, in order to palliate, ameliorate, stabilize, reverse, slow, delay, prevent, reduce, or eliminate either the disease or the symptoms of the disease, or to retard or stop the progression of the disease or of symptoms of the disease. “Therapeutic use” of the polyamines and polyamine analogs is defined as using one or more polyamines or polyamine analogs to treat a disease (including to prevent a disease), as defined above. A “therapeutically effective amount” is an amount sufficient to treat (including to prevent) a disease, as defined above. Prevention or suppression can be partial or total.

As used herein, “suppressing tumor growth” refers to at least partially reducing the rate of growth of a tumor, halting tumor growth completely, causing a regression in the size of an existing tumor, eradicating an existing tumor and/or preventing the occurrence of additional tumors upon treatment with the compositions, kits or methods of the present invention. “Suppressing” tumor growth indicates a growth state that is curtailed when compared to growth by cells treated only with a DNA-damaging agent (e.g., radiation or chemotherapy), without treatment with the siRNA of the invention. Tumor cell growth can be assessed by any means known in the art, including, but not limited to, directly measuring tumor size, radiographic imaging, utilizing serum biomarkers of disease burden (e.g., serum PSA), determining whether tumor cells are proliferating using a ³H-thymidine incorporation assay or clonogenic assay, or counting tumor cells.

As used herein, “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum, tissue, cultured cells, e.g., primary cultures, explants, and transformed cells; stool, urine, ejaculate, or other biological fluids. A biological sample also includes a surgical sample taken from a patient during a surgery, for example, from a tumor or tumor margins.

The term “alkyl” refers to saturated aliphatic groups including straight-chain, branched-chain, cyclic groups, and combinations thereof, having the number of carbon atoms specified, or if no number is specified, having up to 12 carbon atoms. “Straight-chain alkyl” or “linear alkyl” groups refer to alkyl groups that are neither cyclic nor branched, commonly designated as “n-alkyl” groups. C₁-C₈ n-alkyl consists of the following groups: —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH—, —CH₂CH₂CH₂CH₂CH₂CH₂CH—, and —CH₂CH₂CH₂CH₂CH₂CH₂CH₂CH₂—. Other examples of alkyl groups include, but are not limited to, groups such as methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. Cycloalkyl groups can consist of one ring, including, but not limited to, groups such as cycloheptyl, or multiple bridged or fused rings, including, but not limited to, groups such as adamantyl or norbornyl groups. Cycloalkyl groups can also contain alkyl groups in addition to the cyclic portion, e.g., 2,6,6-trimethylbicyclo[3.1.1]heptane, 2-methyldecalin (2-methyldecahydronaphthalene), cyclopropylmethyl, cyclohexylmethyl, cycloheptylmethyl, and the like.

“Substituted alkyl” refers to alkyl groups substituted with one or more substituents including, but not limited to, groups such as halogen (including fluoro, chloro, bromo, and/or iodo-substituted alkyl such as a monohaloalkyl, dihaloalkyl, trihaloalkyl or multihaloalkyl, including a perhalooalkyl, for example, perfluoroalkyl, percholoralkyl, trifluoromethyl or pentachloroethyl), alkoxy, acyloxy, amino (including NH₂, NHalkyl and N(alkyl)₂), hydroxyl, mercapto, carboxy, benzyloxy, phenyl, benzyl, cyano, nitro, acyl, acylamino, amidino, alkyl amidino, thioamidino, aminoacyl, aryl, substituted aryl, aryloxy, azido, thioalkyl, —OS(O)₂-alkyl, thioalkoxy, carboxaldehyde, carboalkoxy and carboxamide, or a functionality that can be suitably blocked, if necessary for purposes of the invention, with a protecting group. Examples of substituted alkyl groups include, but are not limited to, CF₃, CF₂CF₃, and other perfluoro and perhalo groups; —CH₂—OH; —CH₂CH₂CH(NH₂)CH₃, etc. Alkyl groups can be substituted with other alkyl groups, e.g., C₃-C₂₄ cycloalkyl groups.

The term “alkenyl” refers to unsaturated aliphatic groups including straight-chain (linear), branched-chain, cyclic groups, and combinations thereof, having the number of carbon atoms specified, or if no number is specified, having up to 12 carbon atoms, which contain at least one double bond (—C═C—). Examples of alkenyl groups include, but are not limited to, —CH₂—CH═CH—CH₃; and —CH₂—CH₂-cyclohexenyl, where the ethyl group can be attached to the cyclohexenyl moiety at any available carbon valence. The term “alkynyl” refers to unsaturated aliphatic groups including straight-chain (linear), branched-chain, cyclic groups, and combinations thereof, having the number of carbon atoms specified, or if no number is specified, having up to 12 carbon atoms, which contain at least one triple bond (—C≡C—). “Hydrocarbon chain” or “hydrocarbyl” refers to any combination of straight-chain, branched-chain, or cyclic alkyl, alkenyl, or alkynyl groups, and any combination thereof. “Substituted alkenyl,” “substituted alkynyl,” and “substituted hydrocarbon chain” or “substituted hydrocarbyl” refer to the respective group substituted with one or more substituents, including, but not limited to, groups such as halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or any group listed above for “Substituted alkyl,” or a functionality that can be suitably blocked, if necessary for purposes of the invention, with a protecting group.

“Aryl” or “Ar” refers to an aromatic carbocyclic group having a single ring (including, but not limited to, groups such as phenyl), two or more rings connected to each other (including, but not limited to, groups such as biphenyl and p-diphenylbenzene) or two or more condensed rings (including, but not limited to, groups such as naphthyl, anthryl, or pyrenyl), and includes both unsubstituted and substituted aryl groups. Aryls, unless otherwise specified, contain from 6 to 20 carbon atoms in the ring portion. A preferred range for aryls contains 6 to 12 carbon atoms in the ring portion. “Substituted aryls” refers to aryls substituted with one or more substituents, including, but not limited to, groups such as substituted or unsubstituted alkyl, substituted or unsubstituted alkenyl, substituted or unsubstituted alkynyl, substituted or unsubstituted hydrocarbon chains, halogen, alkoxy, acyloxy, amino, hydroxyl, mereapto, carboxy, benzyloxy, phenyl, benzyl, cyano, nitro, thioalkoxy, carboxaldehyde, carboalkoxy and carboxamide, or any group listed above for “Substituted alkyl,” or a functionality that can be suitably blocked, if necessary for purposes of the invention, with a protecting group. “Aralkyl” designates an alkyl-substituted aryl group, where any aryl can be attached to the alkyl; the alkyl portion can comprise one, two, or three straight chains of 1 to 6 carbon atoms each or one, two, or three branched chains of 3 to 6 carbon atoms each or any combination thereof. Aralkyl groups can consist of two aryl groups connected by an alkyl group, such as diphenylmethane or 2-methyl-1-(phenethyl)benzene. When an aralkyl group is indicated as a substituent, the aralkyl group can be connected to the remainder of the molecule at any available valence on either its alkyl moiety or aryl moiety; e.g., the tolyl aralkyl group can be connected to the remainder of the molecule by replacing any of the five hydrogens on the aromatic ring moiety with the remainder of the molecule, or by replacing one of the alpha-hydrogens on the methyl moiety with the remainder of the molecule. Preferably, the aralkyl group is connected to the remainder of the molecule via the alkyl moiety.

An exemplary aryl group is phenyl, which can be substituted or unsubstituted. Substituents for substituted phenyl groups include lower alkyl (—C₁-C₄ alkyl), or a halogen (chlorine (Cl), bromine (Br), iodine (I), or fluorine (F); hydroxy (—OH), or lower alkoxy (—C₁-C₄ alkoxy), such as methoxy, ethoxy, propyloxy (propoxy) (either n-propoxy or i-propoxy), and butoxy (either n-butoxy, i-butoxy, sec-butoxy, or tert-butoxy); a preferred alkoxy substituent is methoxy. Substituted phenyl groups preferably have one or two substituents; more preferably, one substituent.

“Heteroalkyl,” “heteroalkenyl,” and “heteroalkynyl” refer to alkyl, alkenyl, and alkynyl groups, respectively, that contain the number of carbon atoms specified (or if no number is specified, having up to 12 carbon atoms) which contain one or more heteroatoms as part of the main, branched, or cyclic chains in the group. Heteroatoms include, but are not limited to, N, S, O, and P; N and O are preferred. Heteroalkyl, heteroalkenyl, and heteroalkynyl groups may be attached to the remainder of the molecule at any valence where a hydrogen can be removed, for example, at a heteroatom or at a carbon atom (if a valence is available at such an atom by removing a hydrogen). Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₂—, 1-ethyl-6-propylpiperidino, and morpholino. Examples of heteroalkenyl groups include, but are not limited to, groups such as —CH═CH—NH—CH(CH₃)—CH₂—. “Heteroaryl” or “HetAr” refers to an aromatic carbocyclic group having a single ring (including, but not limited to, examples such as pyridyl, imidazolyl, thiophene, or furyl) or two or more condensed rings (including, but not limited to, examples such as indolizinyl, indole, benzimidazole, benzotriazole, or benzothienyl) and having at least one hetero atom, including, but not limited to, heteroatoms such as N, O, P, or S, within the ring. Unless otherwise specified, heteroalkyl, heteroalkenyl, heteroalkynyl, and heteroaryl groups have between one and five heteroatoms and between one and twelve carbon atoms. “Substituted heteroalkyl,” “substituted heteroalkenyl,” “substituted heteroalkynyl,” and “substituted heteroaryl” groups refer to heteroalkyl, heteroalkenyl, heteroalkynyl, and heteroaryl groups substituted with one or more substituents, including, but not limited to, groups such as substituted or unsubstituted alkyl, substituted or unsubstituted alkenyl, substituted or unsubstituted alkynyl, substituted or unsubstituted benzyl, substituted or unsubstituted hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or any group listed above for “substituted alkyl,” or a functionality that can be suitably blocked, if necessary for purposes of the invention, with a protecting group. Examples of such substituted heteroalkyl groups include, but are not limited to, piperazine, substituted at a nitrogen or carbon by a phenyl or benzyl group, and attached to the remainder of the molecule by any available valence on a carbon or nitrogen, —NH—SO₂-phenyl, —NH—(C═O)O-alkyl, —NH—(C═O)O-alkyl-aryl, and —NH—(C═O)-alkyl. If chemically possible, the heteroatom(s) and/or the carbon atoms of the group can be substituted. A “heteroaralkyl” group is a heteroaryl group substituted with at least one alkyl group. The heteroatom(s) can also be in oxidized form, if chemically possible.

The term “alkoxy” as used herein refers to an alkyl, alkenyl, alkynyl, or hydrocarbon chain linked to an oxygen atom and having the number of carbon atoms specified, or if no number is specified, having up to 12 carbon atoms. Examples of alkoxy groups include, but are not limited to, groups such as methoxy, ethoxy, propyloxy (propoxy) (either n-propoxy or i-propoxy), and butoxy (either n-butoxy, i-butoxy, sec-butoxy, or tert-butoxy).

The terms “halo” and “halogen” as used herein refer to the Group VIIa elements (Group 17 elements in the 2005 IUPAC Periodic Table, IUPAC Nomenclature of Inorganic Chemistry) and include Cl, Br, F and I substituents.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al, (1999) Protective Groups in Organic Synthesis, (Wiley-Interscience., New York). Amino protecting groups include, but are not limited to, mesitylenesulfonyl (Mts), benzyloxycarbonyl (CBz or Z), t-butyloxycarbonyl (Boc), t-butyldimethylsilyl (TBS or TBDMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5 bromo 7-nitroindolinyl, and the like. Hydroxyl protecting groups include, but are not limited to, Fmoc, TBS, photolabile protecting groups (such as nitroveratryl oxymethyl ether (Nvom)), Mom (methoxy methyl ether), and Mem (methoxy ethoxy methyl ether), NPEOC (4-nitrophenethyloxycarbonyl) and NPEOM (4 nitrophenethyloxymethyloxycarbonyl).

Various inhibitors of histone demethylase enzymes have been disclosed in U.S. Patent Publication No. 2010/0273745, the entire content of which is herein incorporated by reference.

In one embodiment, the compound is a polyaminoguanidine of the formula (I):

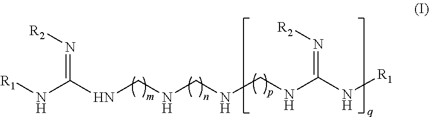

or a salt, solvate, or hydrate thereof, wherein n is an integer from 1 to 12, m and p are independently an integer from 1 to 5, q is 0 or 1, each R₁ is independently selected from the group consisting of C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, and C₇-C₂₄ substituted or unsubstituted heteroaralkyl, and each R₂ is independently selected from hydrogen or a C₁-C₈ substituted or unsubstituted alkyl.

In one embodiment, the compound is of the formula (I) wherein at least one or both R₁ is a C₆-C₂₀ substituted or unsubstituted aryl, such as a single ring substituted or unsubstituted aryl, including without limitation, substituted or unsubstituted phenyl. In one embodiment, the compound is of the formula (I) and each R₁ is phenyl. In one embodiment, q is 1, m and p are 3, and n is 4. In another embodiment, q is 1, m and p are 3, and n is 7.

In one embodiment, the compound is of the formula (I) wherein at least one or both R₁ is a C₈-C₁₂ or a C₁-C₈ substituted or unsubstituted alkyl, such as a linear alkyl. One or both R₁ may be a C₁-C₈ substituted or unsubstituted linear alkyl, such as methyl or ethyl. In one embodiment, each R₁ is methyl. Each or both R₁ may comprise or be a C₄-C₁₅ is cycloalkyl group, such as a cycloalkyl group containing a linear alkyl group, where the cycloalkyl group is connected to the molecule either via its alkyl or cycloalkyl moiety. For instance, each or both R₁ may be cyclopropylmethyl or cyclohexylmethyl. In one embodiment, one R₁ is cyclopropylmethyl or cyclohexylmethyl and the other R₁ is a linear alkyl group, such as a linear C₁-C₈ unsubstituted alkyl group, including without limitation an ethyl group. In one embodiment, R₁ is a C₃-C₁₅ branched alkyl group such as isopropyl. When R₁ is a C₁-C₈ substituted alkyl, the substituted alkyl may be substituted with any substituent, including a primary, secondary, tertiary or quaternary amine. Accordingly, in one embodiment, R₁ is a C₁-C₈ alkyl group substituted with an amine such that R₁ may be e.g., alkyl-NH₂ or an alkyl-amine-alkyl moiety such as —(CH₂)_{y}NH(CH₂)zCH₃ where y and z are independently an integer from 1 to 8. In one embodiment, R₁ is —(CH₂)₃NH₂.

In one embodiment, the compound is of the formula (I) where at least one R₁ is a C₇-C₂₄ substituted or unsubstituted aralkyl, which in one embodiment is an aralkyl connected to the molecule via its alkyl moiety (e.g., benzyl). In one embodiment, each R₁ is an aralkyl moiety wherein the alkyl portion of the moiety is substituted with two aryl groups and the moiety is connected to the molecule via its alkyl group. For instance, in one embodiment at least one or both R₁ is a C₇-C₂₄ aralkyl wherein the alkyl portion is substituted with two phenyl groups, such as when R₁ is 2,2-diphenylethyl or 2,2-dibenzylethyl. In one embodiment, each R₁ of formula (I) is 2,2-diphenylethyl and n is 1, 2 or 5. In one embodiment, each R₁ of formula (I) is 2,2-diphenylethyl, n is 1, 2 or 5 and m and p are each 1.

In one embodiment, at least one R₁ is hydrogen. When at least one R₁ is hydrogen, the other R₁ may be any moiety listed above for R₁, including an aryl group such as benzyl.

Any of the compounds of formula (I) listed above include compounds where at least one or both of R₂ is hydrogen or a C₁-C₈ substituted or unsubstituted alkyl. In one embodiment, each R₂ is an unsubstituted alkyl such as methyl. In another embodiment, each R₂ is hydrogen.

Any of the compounds of formula (I) listed above may be compounds where q is 1 and m and p are the same. Accordingly, the polyaminoguanidines of formula (I) may be symmetric with reference to the polyaminoguanidine core (e.g., excluding R₁). Alternatively, the compounds of formula (I) may be asymmetric, e.g., when q is 0. In one embodiment, m and p are 1. In one embodiment, q is 0. In one embodiment, n is an integer from 1 to 5.

It is understood and clearly conveyed by this invention that each R₁, R₂, m, n, p and q disclosed in reference to formula (I) intends and includes all combinations thereof the same as if each and every combination of R₁, R₂, m, n, p and q were specifically and individually listed.

In one embodiment, the compound is a polyaminobiguanide or N-alkylated polyaminobiguanide. An N-alkylated polyaminobiguanide intends a polyaminobiguanide wherein at least one imine nitrogen of at least one biguanide is alkylated. In one embodiment, the compound is a polyaminobiguanide of the formula (II):

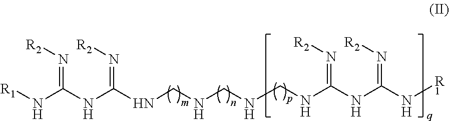

or a salt, solvate, or hydrate thereof, wherein n is an integer from 1 to 12, m and p are independently an integer from 1 to 5, q is 0 or 1, each R₁ is independently selected from the group consisting of C₁-C₈ substituted or unsubstituted alkyl, C₆-C₂₀ substituted or unsubstituted aryl, C₆-C₂₀ substitute or unsubstituted heteroaryl, C₇-C₂₄ substituted or unsubstituted aralkyl, and C₇-C₂₄ substituted or unsubstituted heteroaralkyl and each R₂ is independently hydrogen or a C₁-C₈ substituted or unsubstituted alkyl.

In one embodiment, at least one or each R₁ is a C₁-C₈ substituted or unsubstituted alkyl, such as those listed above in reference to formula (I). For instance, when R₁ is a C₁-C₈ substituted alkyl, the substituted alkyl may be substituted with any substituent, including a primary, secondary, tertiary or quaternary amine. Accordingly, in one embodiment, R₁ is a C₁-C₈ alkyl group substituted with an amine such that R₁ may be e.g., alkyl-NH₂ or an alkyl-amine-alkyl moiety such as —(CH₂)_{y}NH(CH₂)zCH₃ where y and z are independently an integer from 1 to 8. In one embodiment, R₁ is —(CH₂)₃NH₂. R₁ may also be a C₄-C₁₅ substituted or unsubstituted cycloalkyl or a C₃-C₁₅ substituted or unsubstituted branched alkyl, such as described for formula (I) above. In one embodiment, at least one or each R₁ is a C₆-C₂₀ substituted or unsubstituted aryl, such as those listed above in reference to formula (I), In one embodiment, q is 1, m and p are 3, and n is 4. In another embodiment, q is 1, m and p are 3, and n is 7.

In one embodiment, the compound is of the formula (II) where at least one or both R₁ is a C₇-C₂₄ substituted or unsubstituted aralkyl, which in one embodiment is an aralkyl connected to the molecule via its alkyl moiety. In one embodiment, each R₁ is an aralkyl moiety wherein the alkyl portion of the moiety is substituted with one or two aryl groups and the moiety is connected to the molecule via its alkyl moiety. For instance, in one embodiment at least one or both R₁ is an aralkyl wherein the alkyl portion is substituted with two phenyl or benzyl groups, such as when R₁ is 2,2-diphenylethyl or 2,2-dibenzylethyl. In one embodiment, each R₁ of formula (II) is 2,2-diphenylethyl and n is 1, 2 or 5. In one embodiment, each R₁ of formula (II) is 2,2-diphenylethyl and n is 1, 2 or 5 and m and p are each 1.

Any of the compounds of formula (II) listed above include compounds where at least one or both of R₂ is hydrogen or a C₁-C₈ substituted or unsubstituted alkyl. In one embodiment, each R₂ is an unsubstituted alkyl, such as methyl. In another embodiment, each R₂ is a hydrogen.

Any of the compounds of formula (II) listed above include compounds where q is 1 and m and p are the same. Accordingly, the polyaminobiguanides of formula (II) may be symmetric with reference to the polyaminobiguanide core (e.g., excluding R₁). Alternatively, the compounds of formula (II) may be asymmetric, e.g., when q is 0. In one embodiment, m and p are 1. In one embodiment, q is 0. In one embodiment, n is an integer from 1 to 5. In one embodiment, q, m and p are each 1 and n is 1, 2 or 5.

It is understood and clearly conveyed by this invention that each R₁, R₂, m, n, p and q disclosed in reference to formula (II) intends and includes all combinations thereof the same as if each and every combination of R₁, R₂, m, n, p and q were specifically and individually listed.

In one embodiment, the compound is a polyamine. In one embodiment, the polyamine is of the formula (III):

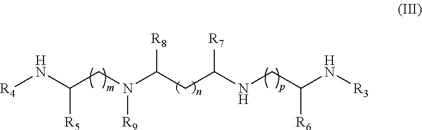

or a salt, solvate, or hydrate thereof, wherein n is an integer from 1 to 12; m and p are independently an integer from 1 to 5; R₃ and R₄ are independently selected from the group consisting of hydrogen, C₁-C₈ substituted or unsubstituted alkyl, C₅-C₂₀, substituted or unsubstituted aryl and C₇-C₂₄ substituted or unsubstituted aralkyl; R₅, R₉, R₆, R₇ and R₈ are independently selected from the group consisting of hydrogen and C₁-C₈ substituted or unsubstituted alkyl; and wherein either m and p are not the same integer or at least one of R₅, R₉, R₆, R₇ and R₈ is a C₁-C₈ substituted or unsubstituted alkyl.

In one embodiment, R₉ is a C₁-C₈ substituted or unsubstituted alkyl. When R₉ is a C₁-C₈ substituted alkyl, the substituted alkyl may be substituted with any substituent, including a primary, secondary, tertiary or quaternary amine. Accordingly, in one embodiment, R₉ is a C₁-C₈ alkyl group substituted with an amine such that R₉ may be e.g., alkyl-NH₂ or an alkyl-amine-alkyl moiety such as —(CH₂)_{y}NH(CH₂)zCH₃ where y and z are independently an integer from 1 to 8. In one embodiment, R₉ is —(CH₂)₃NHCH₂CH₃.

In one embodiment, one or both of R₃ and R₄ is hydrogen. If only one of R₃ and R₄ is hydrogen, the R₃ or R₄ that is not hydrogen may be any moiety described herein, such as a C₁-C₈ substituted or unsubstituted alkyl group, including a cyclic alkyl group such as cyclopropylmethyl or cycloheptylmethyl.

In one embodiment, one or both of R₃ and R₄ is a C₁-C₈ substituted or unsubstituted alkyl, including without limitation a substituted or unsubstituted n-alkyl (such as n-pentyl), substituted or unsubstituted branched (C₃-C₈) alkyl (such as 2-methylbutyl) or substituted or unsubstituted (C₃-C₈) cycloalkyl (such as cyclohexylmethyl). Larger chain alkyl (linear, branched and cyclic) are also considered, such as a C₉-C₁₅ substituted or unsubstituted alkyl. Where one or both of R₃ and R₄ is a C₁-C₈ substituted or unsubstituted n-alkyl, the moiety may be any n-alkyl, such as methyl or ethyl. In one embodiment, both R₃ and R₄ are a C₁-C₈ substituted or unsubstituted alkyl, wherein one of R₃ and R₄ is an n-alkyl moiety and the other is a cyclic moiety, which is understood to contain at least three carbon atoms. Alternatively, both R₃ and R₄ may be a C₁-C₈ substituted or unsubstituted n-alkyl. When one or both of R₃ and R₄ is a substituted alkyl, whether linear, branched or cyclic, the alkyl may be substituted with one or more substituents such as those listed under “Substituted alkyl” and includes alkyl substituted with any halogen, such as a monohaloalkyl, dihaloalkyl, trihaloalkyl or multihaloalkyl, including a perhalooalkyl, for example, perfluoroalkyl and percholoralkyl, such as trifluoromethyl or pentachloroethyl.

In one embodiment, one or both of R₃ and R₄ is a C₆-C₂₀ substituted or unsubstituted aryl. In one embodiment, one or both of R₃ and R₄ is a C₆-C₂₀ substituted aryl, which aryl groups may be substituted with one or more substituents such as those listed under “Substituted aryl.” In one embodiment, one or both of R₃ and R₄ is a C₆-C₂₀ substituted aryl, which aryl groups may be substituted with one or more alkyoxy (such as —OCH₃), alkyl (including a branched alkyl such as tert-butyl), or halo groups (such as fluoro). In one embodiment, one or both of R₃ and R₄ is a halo-substituted aryl or a halo-substituted aralkyl, such as 2,4,5-trifluorophenyl or 2,4,5-trifluorobenzyl. In one embodiment, one or both of R₃ and R₄ is a di-alkyl-monoalkoxy-substituted aryl or aralkyl, such as 4,5-di-tert-butyl-2-methoxybenzyl or 4,5-di-tert-butyl-2-methoxyphenyl.

In one embodiment, one or both of R₃ and R₄ is a C₇-C₂₄ substituted or unsubstituted aralkyl or heteroaralkyl such as an aralkyl or heteroaralkyl connected to the molecule via its alkyl moiety. In one embodiment, one or both of R₃ and R₄ is a substituted aralkyl or heteroaralkyl connected to the molecule via its alkyl moiety. A substituted aralkyl may be substituted with one or more substituents such as those listed under “Substituted aralkyl” and a substituted heteroaralkyl may be substituted with one or more substituents such as those listed under “substituted heteroaralkyl.” In one embodiment, one or both of R₃ and R₄ is a substituted heteroaralkyl having at least one nitrogen atom. In one embodiment, one or both of R₃ and R₄ is a single ring heteroaralkyl having at least one nitrogen atom. In one embodiment, one or both of R₃ and R₄ is 1-(2-N-methylpyrrolyl)-methyl.

In one embodiment, at least 1 or at least 2 or at least 3 of R₅, R₉, R₆, R₇ and R₈ is a C₁-C₈ substituted or unsubstituted alkyl. R₅, R₉, R₆, R₇ and R₈ may be a C₁-C₈ substituted or unsubstituted alkyl. In one embodiment at least 1 or at least 2 or at least 3 of R₅, R₉, R₆, R₇ is a C₁-C₈ unsubstituted n-alkyl, such as methyl or ethyl. In one embodiment, both R₆ and R₅ are methyl or ethyl. In one embodiment, at least one R₇ and R₈ is methyl or ethyl. In one embodiment, R₇ is methyl.

It is understood and clearly conveyed by this invention that each R₃, R₄, R₅, R₉, R₆, R₇, R₈, m, n, y, z and p disclosed in reference to formula (III) intends and includes all combinations thereof the same as if each and every combination of R₃, R₄, R₅, R₉, R₆, R₇, R₈, m, n, y, z and p were specifically and individually listed.

In one embodiment, the polyamine is of the formula (IV):

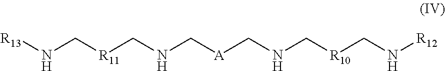

or a salt, solvate, or hydrate thereof, wherein A, R₁₀ and R₁₁ are independently (CH₂)ₙ or ethene-1,1-diyl; n is an integer from 1 to 5; R₁₂ and R₁₃ are independently selected from the group consisting of hydrogen, C₂-C₈ substituted or unsubstituted alkenyl and C₁-C₈ substituted or unsubstituted alkyl; and at least one of A, R₁₀, R₁₁, R₁₂ and R₁₃ comprises an alkenyl moiety. In another embodiment, when any one or more of A, R₁₀, and R₁₁ is alkenyl, the alkene portion branches off the direct chain connecting the nitrogen atoms; that is, no more than one sp²-hybridized carbon occurs in the carbon nodes along the shortest path from one nitrogen flanking A, R₁₀, and/or R₁₁ to the other flanking nitrogen. For example, when A is ethene, the segment containing A is of the form —CH₂C(═CH₂)—CH₂— and the three nodes in the shortest carbon path between the nitrogens containing the A moiety has only one sp²-hybridized carbon. When A is propene, the segment containing A can be of the form —CH₂C(═CHCH₃)—CH₂— or —CH₂C(═CH═CH₂)—CH₂—.

In one embodiment, A is (CH₂)ₙ and n is 1. In one embodiment, A is ethene-1,1-diyl. In one embodiment, A is (CH₂)ₙ and one or both of R₁₂ and R₁₃ comprises an alkenyl moiety, such as propen-2-yl.

In one embodiment at least one or both of R₁₀ and R₁₁ is ethene-1,1-diyl. In one embodiment, both R₁₀ and R₁₁ are (CH₂)ₙ such as CH₂ (where n=1).

In one embodiment, at least one or both of R₁₂ and R₁₃ is hydrogen. In one embodiment, at least one or both of R₁₂ and R₁₃ is a C₂-C₈ substituted or unsubstituted alkenyl, such as propen-2-yl. In one embodiment, at least one or both of R₁₂ and R₁₃ is a C₁-C₈ substituted or unsubstituted alkyl, such as methyl or ethyl or any C₁-C₈ substituted or unsubstituted alkyl mentioned above in reference to any one of formulae (I), (II) or (III).

It is understood and clearly conveyed by this invention that each A, n, R₁₀, R₁₁, R₁₂ and R₁₃ disclosed in reference to formula (IV) intends and includes all combinations thereof the same as if each and every combination of A, n, R₁₀, R₁₁, R₁₂ and R₁₃ were specifically and individually listed.

In one embodiment, the polyamine is of the formula (V):

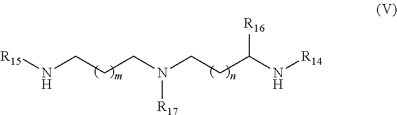

or a salt, solvate, or hydrate thereof, wherein n is an integer from 1 to 8; m is an integer from 1 to 8; R₁₅ and R₁₄ are independently selected from the group consisting of hydrogen, C₁-C₈ substituted or unsubstituted n-alkyl or (C₃-C₈) branched alkyl, C₆-C₂₀ substituted or unsubstituted aryl or heteroaryl and C₇-C₂₄ substituted or unsubstituted aralkyl or heteroaralkyl; R₁₆ and R₁₇ are independently hydrogen or a C₁-C₈ substituted or unsubstituted alkyl; and wherein the compound contains no more than three secondary amino groups except when R₁₇ is a C₁-C₈ substituted or unsubstituted alkyl and wherein the compound is free from a methylphosphonate or hydroxy moiety.

In one embodiment, at least one or both of R₁₅ and R₁₄ is hydrogen. When only one of R₁₅ and R₁₄ is hydrogen, the R₁₅ or R₁₄ that is not hydrogen may be any other moiety listed above, such as a C₆-C₂₀ substituted or unsubstituted aryl or heteroaryl (e.g.; 4-isopropylbenzyl, 2-phenylbenzyl, 3,3,-diphenylpropyl and the like or any C₆-C₂₀ substituted or any unsubstituted aryl or heteroaryl listed above in reference to any one of formulae (I)-(IV)).

In one embodiment, at least one or both of R₁₅ and R₁₄ is a C₁-C₈ substituted or unsubstituted n-alkyl or (C₃-C₈) branched alkyl, such as methyl, ethyl, 3-methyl-butyl, 2-ethyl-butyl, 5-NH₂-pent-1-yl, prop-1-yl-methyl(phenyl)phosphinate and the like or any C₁-C₈ substituted or unsubstituted n-alkyl or (C₃-C₈) branched alkyl listed above in reference to formulae (I)-(IV). In one embodiment, at least one or both of R₁₅ and R₁₄ is a C₁-C₈ substituted or unsubstituted n-alkyl, such as an n-alkyl substituted with a methyl(phenyl)phosphinate moiety or a NH₂-substituted n-alkyl. In one embodiment, both R₁₅ and R₁₄ are C₁-C₈ substituted or unsubstituted n-alkyl or (C₃-C₈) branched alkyl moieties, such as when R₁₅ and R₁₄ are both 3-methyl-butyl or when R₁₅ and R₁₄ are both 2-ethyl-butyl. R₁₅ and R₁₄ may be different C₁-C₈ substituted or unsubstituted n-alkyl moieties, such as when one of R₁₅ and R₁₄ is propyl and the other is ethyl.

In one embodiment, at least one or both of R₁₅ and R₁₄ is a C₇-C₂₄ substituted or unsubstituted aralkyl or heteroaralkyl. In one embodiment, at least one or both of R₁₅ and R₁₄ is a C₇-C₂₄ substituted or unsubstituted aralkyl or heteroaralkyl having two rings, such as 2-phenylbenzyl, 4-phenylbenzyl, 2-benzylbenzyl, 3-benzylbenzyl, 3,3,-diphenylpropryl, 3-(benzoimidazolyl)-propyl and the like. In one embodiment, at least one or both of R₁₅ and R₁₄ is a C₇-C₂₄ substituted or unsubstituted aralkyl or heteroaralkyl having one ring, such as 4-isopropylbenzyl, 4-fluorobenzyl, 4-tert-butylbenzyl, 3-imidazolyl-propyl, 2-phenylethyl and the like. In one embodiment, one of R₁₅ and R₁₄ is a C₇-C₂₄ substituted or unsubstituted aralkyl or heteroaralkyl, such as any of the specific substituted or unsubstituted aralkyl or heteroaralkyl moieties listed for any other formula, and the other R₁₅ and R₁₄ is hydrogen or a C₁-C₈ substituted or unsubstituted n-alkyl or (C₃-C₈) branched alkyl, such as ethyl, methyl, 3-methylbutyl and the like.

For any compound of formula (V), m and n may be the same or different. In one embodiment, m does not equal n, such as when m is 1 and n is 2. For instance, in one embodiment, m is 1, n is 2 and both R₁₅ and R₁₄ are 2-benzylbenzyl. However, it is understood that all possible combinations of m, n, R₁₅ and R₁₄ are intended.

In one embodiment, at least one or both of R₁₆ and R₁₇ is hydrogen. In one embodiment, at least one or both of R₁₆ and R₁₇ is a C₁-C₈ substituted or unsubstituted alkyl, such as a methyl, ethyl and a C₁-C₈ alkyl substituted with e.g., an —NH—C₁-C₈ alkyl such as when at least one or both of R₁₆ and R₁₇ is —CH₂)₃NHCH₂CH₃.

It is understood and clearly conveyed by this invention that each R₁₄, R₁₅, R₁₆, R₁₇, m, and n disclosed in reference to formula (V) intends and includes all combinations thereof the same as if each and every combination of R₁₄, R₁₅, R₁₆, R₁₇, m, and n were specifically and individually listed.

In one embodiment, the polyamine is of the formula (VI):

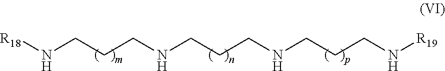

or a salt, solvate, or hydrate thereof, wherein n is an integer from 1 to 12; m and p are independently an integer from 1 to 5; R₁₈ and R₁₉ are independently selected from the group consisting of hydrogen, C₁-C₈ unsubstituted alkyl (e.g., methyl, ethyl, Cert-butyl, isopropyl, pentyl, cyclobutyl), C₁-C₈ n-alkyl substituted with a cycloalkyl group comprising at least two rings, C₇-C₂₄ substituted or unsubstituted aralkyl or heteroaralkyl comprising at least two rings; and wherein: n is 1 when R₁₈ and R₁₉ are identical C₁-C₈ n-alkyl moieties substituted with a cycloalkyl group comprising at least two rings, or are identical aryl groups comprising at least two rings; and, at least one of R₁₈ and R₁₉ is either a C₁-C₈ n-alkyl substituted with a cycloalkyl group comprising at least two rings or a C₇-C₂₄ substituted or unsubstituted aralkyl comprising at least two rings.

In one embodiment, at least one or both of R₁₈ and R₁₉ is a C₁-C₈ n-alkyl substituted with a cycloalkyl group comprising at least two rings. The cycloalkyl group comprising at least two rings may be a spiro, fused or bridged cycloalkyl group. Representative examples of a C₁-C₈ n-alkyl substituted with a cycloalkyl group comprising two rings include moieties such as 2-(6,6-dimethylbicyclo[3.1.1]heptyl)ethyl and 2-(decahydronaphthyl)ethyl. In one embodiment, both R₁₈ and R₁₉ are 2-(6,6-dimethylbicyclo[3.1.1]heptyl)ethyl. In one embodiment, both R₁₈ and R₁₉ are 2-(decahydronaphthyl)ethyl. In one embodiment, one of R₁₈ and R₁₉ is 2-(6,6-dimethylbicyclo[3.1.1]heptyl)ethyl or 2-(decahydronaphthyl)ethyl and the other R₁₈ and R₁₉ is hydrogen or a C₁-C₈ unsubstituted alkyl such as ethyl.

In one embodiment, at least one or both of R₁₈ and R₁₉ is a C₇-C₂₄ substituted or unsubstituted aralkyl or heteroaralkyl comprising at least two rings, which rings may be but are not required to be fused. A substituted aralkyl or heteroaralkyl with reference to formula (VI) intends and includes alkanoyl moieties substituted with an aryl or heteroaryl group, i.e., —C(═O)-aryl, —C(═O)-heteroaryl, and —C(═O)-heteroaralkyl. In one embodiment, the alkyl portion of the aralkyl or heteroaralkyl moiety is connected to the molecule via its alkyl moiety. For instance at least one or both of R₁₈ and R₁₉ may be an aralkyl moiety such as 2-phenylbenzyl, 4-phenylbenzyl, 3,3,-diphenylpropyl, 2-(2-phenylethyl)benzyl, 2-methyl-3-phenylbenzyl, 2-napthylethyl, 4-(pyrenyl)butyl, 2-(3-methylnapthyl)ethyl, 2-(1,2-dihydroacenaphth-4-yl)ethyl and the like. In another embodiment, at least one or both of R₁₈ and R₁₉ may be a heteroaralkyl moiety such as 3-(benzoimidazolyl)propanoyl, 1-(benzoimidazolyl)methanoyl, 2-(benzoimidazolyl)ethanoyl, 2-(benzoimidazolyl)ethyl and the like.

In one embodiment, each of m, n and p is the same, such as when m, n and p are each 1.

It is understood and clearly conveyed by this invention that each R₁₈, R₁₉, m, n and p disclosed in reference to formula (VI) intends and includes all combinations thereof the same as if each and every combination of R₁₈, R₁₉, m, n and p were specifically and individually listed.

In one embodiment, the polyamine is of the formula (VII):

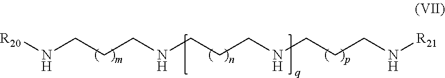

or a salt, solvate, or hydrate thereof, wherein n is an integer from 1 to 12; m and p are independently an integer from 1 to 5; q is 0 or 1; R₂₀ and R₂₁ are independently selected from the group consisting of hydrogen, C₁-C₈ substituted or unsubstituted alkyl, —C(═O)—C₁-C₈ substituted or unsubstituted alkyl, —C(═O)—C₁-C₈ substituted or unsubstituted alkenyl, —C(═)—C₁-C₈ substituted or unsubstituted alkynyl, and C₇-C₂₄ substituted or unsubstituted aralkyl; and wherein the compound comprises at least one moiety selected from the group consisting of t-butyl, isopropyl, 2-ethylbutyl, 1-methylpropyl, 1-methylbutyl, 3-butenyl, isopent-2-enyl, 2-methylpropan-3-olyl, ethylthiyl, phenylthiyl, propynoyl, 1-methyl-1H-pyrrole-2-yl, trifluoromethyl, cyclopropanecarbaldehyde, halo-substituted phenyl, nitro-substituted phenyl, alkyl-substituted phenyl, 2,4,6-trimethylbenzyl, halo-S-substituted phenyl (such as para-(F₃S)-phenyl, azido and 2-methylbutyl.

In one embodiment, q is 1. In one embodiment, q is 1 and n is 1.

In one embodiment at least one of R₂₀ and R₂₁ is hydrogen. In one embodiment at least one of R₂₀ and R₂₁ is C₁-C₈ substituted or unsubstituted alkyl, such as any of the substituted or unsubstituted alkyl moieties mentioned above for formulas (I)-(VI). In one embodiment at least one of R₂₀ and R₂₁ is a C₇-C₂₄ substituted or unsubstituted aralkyl, such as any of the C₇-C₂₄ substituted or unsubstituted aralkyl mentioned above for formulas (I)-(VI).

It is understood and clearly conveyed by this invention that each R₂₀, R₂₁, m, n, q and p disclosed in reference to formula (VII) intends and includes all combinations thereof the same as if each and every combination of R₂₀, R₂₁, m, n, q and p were specifically and individually listed.

In one embodiment, the polyamine is of the formula (VIII):

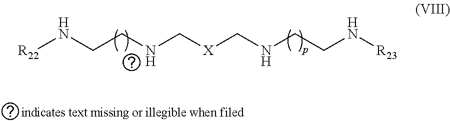

or a salt, solvate, or hydrate thereof, wherein m and p are independently an integer from 1 to 5; X is —(CH₂)ₙ— or cyclohex-1,3-diyl; n is an integer from 1 to 5; R₂₂ and R₂₃ are independently selected from the group consisting of hydrogen, n-butyl, ethyl, cyclohexylmethyl, cyclopentylmethyl, cyclopropylmethyl, cycloheptylmethyl, cyclohexyleth-2-yl, and benzyl; and when n is 5, at least one of R₂₂ and R₂₃ is hydrogen; when R₂₂ is ethyl, R₂₃ is hydrogen, n-butyl, cyclopentylmethyl, cyclohexyleth-2-yl or benzyl; and when R₂₃ is ethyl, Rₙ is hydrogen, n-butyl, cyclopentylmethyl, cyclohexyleth-2-yl or benzyl; when X is cyclohex-1,3-diyl, R₂₂ and R₂₃ are not both benzyl or cyclopropylmethyl.

In one embodiment, X is —(CH₂)ₙ— (e.g., CH₂ where n is 1). In one embodiment, X is CH₂ and m and p are both 1. In one embodiment, X is cyclohex-1,3-diyl. In one embodiment, X is cyclohex-1,3-diyl and m and p are both 1. In other embodiments, m and p are not the same, e.g., when m is 3 and p is 4.

It is understood and clearly conveyed by this invention that each R₂₂, R₂₃, m, n and p disclosed in reference to formula (VIII) intends and includes all combinations thereof the same as if each and every combination of R₂₂, R₂₃, m, n and p were specifically and individually listed.

In one embodiment, the polyamine is of the formula (IX):

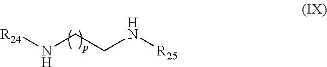

or a salt, solvate, or hydrate thereof, wherein p is an integer from 1 to 5; R₂₄ is an amino-substituted cycloalkyl (e.g., a cycloalkyl group substituted with a primary, secondary, tertiary or quaternary amine) or a C₂-C₈ substituted or unsubstituted alkanoyl (which substituted alkanoyl may be substituted with one or more substituents such as those listed for “Substituted alkyl” including without limitation an alkanoyl substituted with a methyl and an alkylazide group); and R₂₅ is a C₁-C₈ substituted or unsubstituted alkyl or a C₇-C₂₄ substituted or unsubstituted aralkyl, such as those listed above for any of formulae (I)-(VIII).

In one embodiment, R₂₄ is an amino-substituted C₃-C₂₄ cycloalkyl, such as 5-NH₂-cycloheptyl, 3-NH 2-cyclopentyl and the like. In one embodiment, R₂₅ is a C₁-C₈ substituted or unsubstituted alkyl, which includes an n-alkyl group substituted with a cycloalkyl, such as in cyclopropylmethyl. In one embodiment, R₂₅ is cyclopropylmethyl or ethyl and R₂₄ is 5-NH₂-cycloheptyl or 3-NH₂-cyclopentyl. In one embodiment, R₂₄ is a C₂-C₈ substituted or unsubstituted alkanoyl and R₂₄ is a C₇-C₂₄ substituted or unsubstituted aralkyl, such as 4-phenylbenzyl.

It is understood and clearly conveyed by this invention that each R₂₄, R₂₅ and p disclosed in reference to formula (IX) intends and includes all combinations thereof the same as if each and every combination of R₂₄, R₂₅ and p were specifically and individually listed.

For all formulae listed herein, such as formulae (I)-(IX), even if not explicitly stated, any substituent mentioned in one formula is intended to describe the same substituent in any other formula to the extent that the description conforms to the structural characterization of the formula described. For example. R₁ in formula I is intended to describe any other R₁ found in any other formula to the extent that the description conforms to the structural characterization of the formula described. Similarly, any description of, e.g., C₁-C₈ substituted or unsubstituted alkyl is intended to describe any other C₁-C₈ substituted or unsubstituted alkyl found in any other formula to the extent that the description conforms to the structural characterization of the formula described.

It is also recognized that any compounds listed as a particular salt thereof is not intended to limit the compound to such salt or form thereof. Similarly, where compounds are listed as a salt, the structure may or may not explicitly indicate positive or negative charges or the location thereof, and all possibilities thereof are intended. For instance, a compound listed as a 4HBr salt does not limit the compound to only the HBr salt and the compound may or may not show the + or − charges of the HBr salt, but rather all possibilities are intended.

Any of the polyamine compounds, such as compounds of the formula (I)-(IX) may be in a protected form, such as when any one or more amine (e.g., —NH—) is protected by a protecting group (Pg), such as in (—NPg-). Pg may be any protecting group, such as mesityl (e.g., NMes), Boc (e.g., —NBoc) or any other protecting group such as those described in, e.g. T. W. Green, P. G. M. Wuts, Protective Groups in Organic Synthesis, Wiley-Interscience, New York, 1999, which is incorporated herein by reference in its entirety.

Synthetic Methods—Synthesis of Alkylpolyamines: Several synthetic methods are available for synthesis of polyamine analog compounds, including both symmetrically-substituted and asymmetrically-substituted polyamine analogs. Some of these methods are described in the following publications: Saab et al., J. Med. Chem. 36:2998 (1993); Bellevue et al., Bioorg. Med. Chem. Lett. 6:2765 (1996); Sirisoma et al., Tetrahedron Lett. 39:1489 (1998); Zou et al., Bioorg. Med. Chem. Lett. 11:1613 (2001), and Casero et al., J. Med. Chem. 44:1 (2001).

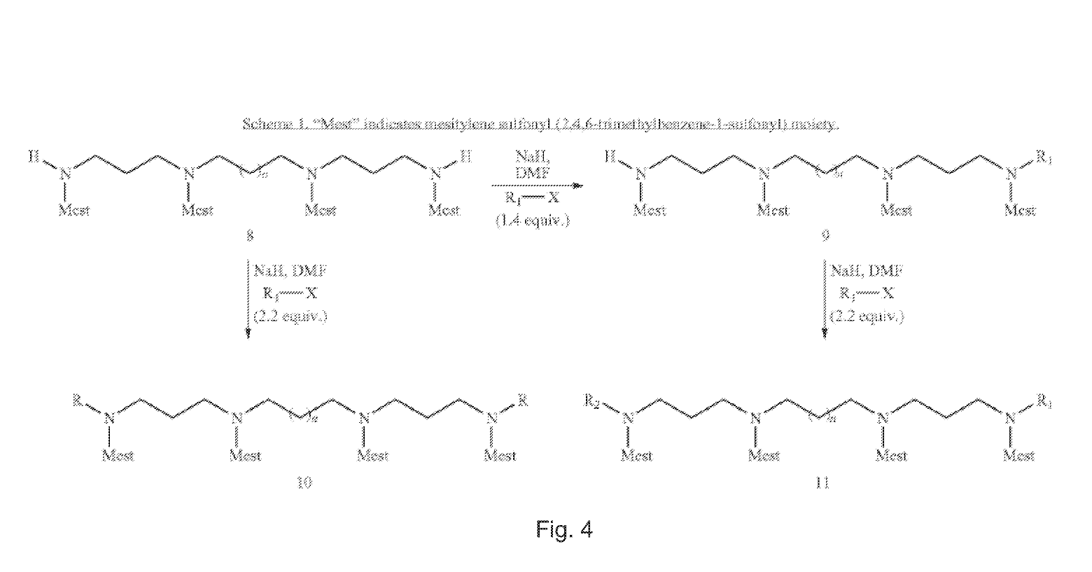

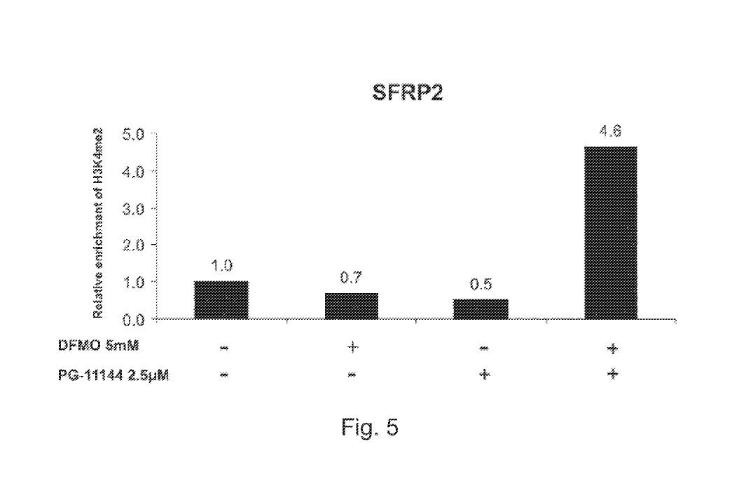

FIG. 4 illustrates a useful pathway to various polyamine analogs. The tetramesitylated intermediate 8 can be readily alkylated at both terminal nitrogens, since the hydrogens on these nitrogens are rendered acidic by the adjacent mesityl protecting group. Alkylation in the presence of 1.2 to 1.4 equivalents of alkyl halide or tosylate affords primarily the monosubstituted product 9, and disubstituted materials and unreacted starting material can then be separated and recycled (Bellevue et al., Bioorg. Med. Chem. Lett. 6:2765 (1996); Zou et al., Bioorg. Med. Chem. Lett. 11:1613 (2001)). The resulting monoalkylated derivative 9 can then be deprotected (30% HBr in AcOH), or realkylated with a different alkyl halide to provide the asymmetrically substituted intermediate 11. Deprotection of 11 then provides the desired asymmetrically substituted alkylpolyamine. Treatment of 8 with 2.2 equivalents of alkyl halide in the presence of NaH and DMF affords the bis-substituted intermediate 10, which upon deprotection yields the corresponding symmetrically substituted alkylpolyamine. Thus three distinct alkylpolyamines can be readily synthesized from a single intermediate, and the central carbon chain can be made in any desired length (n=0-8). Synthesis of the intermediate 8 is readily accomplished in large quantities using previously reported synthetic strategies (Bellevue et al., Bioorg. Med. Chem. Lett. 6:2765 (1996); Zou et al., Bioorg. Med. Chem. Lett. 11:1613 (2001)). A similar strategy can be used to access spermidine-like analogs of formula (XI):

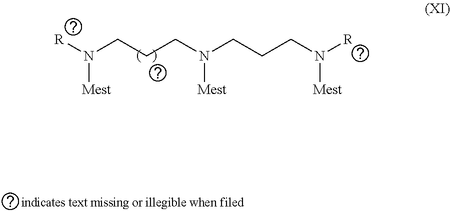

Aminopropyl (or other aminoalkyl) moieties can be added to selectively protected primary amines by standard peptide coupling techniques (Method A, Woster et al., J. Med. Chem. 32:1300 (1989)). Thus treatment with the protected beta-aminopropionate (DCC, HoBt, N-methylmorpholine) affords the corresponding amide, which is then reduced in the presence of diborane (Woster et al., 1989) to afford the desired secondary amine. Compound may be synthesized directly by reductive amination, in which the appropriate aldehyde is added in the presence of sodium cyanoborohydride. Alkyl substituents that contain an allylic acetate functionality can also be appended using a palladium catalyzed coupling reaction that proceeds with retention of configuration (Method C, Sirisoma et al., Tetrahedron Lett. 39:1489 (1998)). This method can also be used to introduce phthalimide or benzylamine to an allylic acetate site as a synthetic equivalent for nitrogen. These nitrogens can then be deprotected and functionalized.

Synthesis of polyaminoguanidines: The requisite amine (produced when necessary from the corresponding alkyl or aralkylcyanide) is reacted with cyanogen bromide (Goldin et al., U.S. Pat. No. 6,288,123 (2001)) to afford the corresponding aminocyanogen. When the desired amine is not commercially available, it can be prepared from the appropriate cyano compound by catalytic reduction (Bellevue et al., 1996, Zou et al., 2001). Intermediate (Bellevue et al., 1996; Zou et al., 2001) is then coupled (chlorobenzene, reflux), followed by deprotection (30% Hbr in AcOH) to produce alkylpolyaminoguanidines. Using these methods, substituted polyaminoguanidine analogs (e.g., R═H, methyl, ethyl, cyclopropylmethylene, cycloheptylmethylene, phenyl, benzyl) can be synthesized. An analogous route (not shown) utilizing the N-Boc protection group was also employed.

The synthesis of polyaminobiguanides is described in Bi et al., Bioorg. Med. Chem. Lett. 16:3229 (2006). Similar strategy is employed for the synthesis of alkylpolyaminobiguanides. Amines (produced when necessary from the corresponding alkyl or aralkylcyanide) are converted to the corresponding cyanoguanidines (NaN(CN)₂, BuOH/H₂0) (Gerhard, R.; Heinz, B.; Herbert, F. J. Praktische Chem. (Leipzig), 1964, 26, 414-418), which were combined to afford the mesityl protected target molecules. Deprotection as described above then provided the substituted biguanides. An analogous route utilizing the N-Boc protection group was also employed.

Solid phase synthetic techniques can be used for the rapid and efficient synthesis of both alkylpolyamines and their alpha-methyl homologs. Compound can be produced using a commercially available trityl chloride resin, as described in Wang et al., J. Am. Chem. Soc., 95(4): 1328 (1973), where the attached amine is primary or secondary prior to attachment, an alpha-methyl is present or absent, and the X group is either a protected amine or a synthetic equivalent such as an azide or a phthalamide. This intermediate is then deprotected or converted to the corresponding primary amine. Three strategies can be used for chain elongation: 1. reductive amination with aldehydes in the presence of sodium cyanoborohydride; 2. addition of an appropriate carboxylate under peptide coupling conditions (Woster et al., J. Med. Chem. 32:1300 (1989)), followed by diborane reduction of the resulting amide; 3. direct alkylation with a protected halide (Woster et al., J. Med. Chem. 32:1300 (1989)). Repetition of these steps then allows the synthesis of a variety of alkylpolyamines and alpha-methyl-alkylpolyamines with substituents as desired.

The invention includes all salts of the compounds described herein. The invention also includes all non-salt compounds of any salt of a compound named herein, as well as other salts of any salt of a compound named herein. In one embodiment, the salts of the compounds comprise pharmaceutically acceptable salts. Pharmaceutically acceptable salts are those salts which retain the biological activity of the free compounds and which can be administered as drugs or pharmaceuticals to humans and/or animals. The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include; but are not limited to, procaine, dibenzylamine, N-ethylpiperidine, N,N′-dibenzylethylenediamine, and triethylamine salts. Salts of acidic compounds with amino acids, such as lysine salts, can also be prepared.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Thus, the term “acid addition salt” refers to the corresponding salt derivative of a parent compound Eat has been prepared by the addition of an acid. The pharmaceutically acceptable salts include the conventional salts or the quaternary ammonium salts of the parent compound formed, for example, from inorganic or organic acids. For example, such conventional salts include, but are not limited to, those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Certain acidic or basic compounds of the present invention may exist as zwitterions. All forms of the compounds, including free acid, free base, and zwitterions, are contemplated to be within the scope of the present invention.

The invention includes all solvates of the compounds described herein, such as hydrates (in any ratios, e.g. monohydrates, dihydrates, hemihydrates, sesquihydrates), methanolates, ethanolates, etc.

Any compound described herein may occur in a combined salt and solvate form, for example the hyclate (monohydrochloride hemiethanolate hemihydrate) form.

The invention includes all stereoisomers of the compounds described herein, including diastereomers and enantiomers in optically pure or substantially optically pure form, as well as mixtures of stereoisomers in any ratio, including, but not limited to, racemic mixtures. Unless stereochemistry is explicitly indicated in a chemical structure or chemical name, the chemical structure or chemical name is intended to embrace all possible stereoisomers of the compound depicted.

The invention includes all crystal and non-crystalline forms of the compounds described herein, including all polymorphs, polycrystalline, and amorphous forms and any mixtures thereof.

Biological Applications-Lysine-Specific Demethylase-1 (LSD1) Inhibitors: Histones are proteins found in eukaryotic cells which act as support scaffolds for DNA (sometimes compared to a protein spool supporting the DNA thread). Histones, together with other proteins and DNA, form the chromatin of the cell nucleus. Because of their close association with DNA, histones play a role in gene regulation. The tails of histone proteins are a frequent site for covalent modifications which affect gene expression.

The enzyme lysine-specific demethylase-1 (LSD1; also known as BHC110 and KIAA0601) is an enzyme that affects the covalent modification of histone tails, by demethylating lysine 4 of the histone H3. Shi et al. (Cell, 119:941 (2004)) showed that RNAi inhibition of LSD1 led to an increase in H3 lysine 4 methylation, followed by de-repression of the target genes. Thus LSD1 apparently represses transcription by demethylating histone H3. Conversely, inhibition of LSD1 allows transcription by preventing demethylation.

Because of the observed homology between the active site of LSD1 and monoamine oxidase (MAO), Lee et al. (Chemistry & Biology 13:563 (2006)) tested various MAO inhibitors for their ability to inhibit LSD1. They identified tranylcypromine ((1R,2S)-2-phenylcyclopropan-1-amine) as an inhibitor with an IC₅₀ less than 2 micromolar. Treating P19 embryonal carcinoma cells with tranylcypromine led to transcriptional de-repression of the Egr1 and Oct4 genes.

International Patent Application WO 2006/071608 is directed to a method for monitoring eukaryotic histone demethylase activity, methods for up-regulating and down-regulating methylated histone-activated genes, and a method for treating or preventing a disease (e.g., a hyperproliferative disease such as cancer) by modulating the level of protein or the activity of a histone demethylase, and the content of which is incorporated by reference in its entirety.

MTT dose response experiments in 235, MCF7, 435, and 10A cells can be performed. MTT is a standard colorimetric assay used for measuring metabolic activity in cells. Briefly, about 200 μl of media not containing cells was added to column A of a 96 well plate and used as a blank. Next, 200 μl of media containing cells was added to the remaining wells and incubated overnight. The remaining wells contain about 4000-5000 MCF7 cells/well, 3000 231 cells/wells, 12,000 468 cells/well, or 9000 MCF 10A cells/well. Following incubation, the media in the wells was aspirated and replaced with 200 μl of fresh media in columns A and B of the 96 well plate. Column B was used as a control. Next 200 μl of fresh media containing the compound being tested was added to the remaining wells and incubated for 96 hours. Compounds can be routinely tested at concentrations ranging from 0.1 micromolar to 50 micromolar. Following incubation for 96 hours, the media in each well was aspirated and replaced with 100 μl of 5 mg/ml MTT (3-(4,5-Dimethylthiazol-2-yl)-2,5-diphenyltetrazolium bromide) solution in Serum-Free media and incubated for 4 hours. Following incubation with MTT solution, the MTT solution was removed from the wells and replaced with 200 ul of a 1:1 Etoh+DMSO solution and incubated for 20 minutes. Following incubation with the Etoh+DMSO solution the plates were read at 540 nm and used to determine the metabolic activity of the cells in the presence of the test compound, relative to the control. IC₅₀ values for the test compounds can be extracted based on the results.

A detailed protocol for determining SSAT activity is described in Casero et al., Cancer Research, 49:3829 (1989). Briefly, the SSAT activity was measured by harvesting the treated cells at the exposure time. The cells were then lysed and treated with spermidine, and 1-[¹⁴C]acetyl coenzyme A for 5 minutes. Enzyme activity was measured in term of picomoles of [¹⁴C]acetylspermidine formed per mg of cell protein per min (pmol/mgP/min).

A detailed protocol for measuring SMO activity is described in Wang et al., Cancer Research, 61:5370 (2001). The compound identifier, the treatment concentration, the control activity, the activity following treatment and the exposure time are listed in columns 1, 2, 3, 4, and 5 respectively. The activity results are reported in picomoles of spermine converted per mg of cell protein per min (pmolimgP/min).

A detailed protocol for measuring ODC activity is described in Pegg et al., Methods Enzymology, 94:158 (1983).

Following exposure of the cells to a compound of interest, at a concentration of, for example, 10 μM, for 24 hours, the cells can be harvested, prepared and transferred to a FACS for cell cycle analysis. (See Carlisle et al., Clinical Cancer Research 8:2684 (2002) and references therein).

2-Difluoromethylornithine (DFMO) is an irreversible inhibitor of ornithine decarboxylase (ODC), the key enzyme in mammalian polyamine biosynthesis. The two enantiomers of DFMO have been reported to differ in their ability to inhibit ODC, with the L form being more potent then the D enantiomer. Although the physiologic functions of polyamines are not completely understood, it is clear that their intracellular concentration is highly regulated and that normal cell growth, replication, differentiation, secretory and repair functions require polyamines. Polyamines have been found in high levels in many tumor cells and support sustained cell growth that is essential for the multistep process of cancer development. In animal models of colon carcinogenesis, inhibition of ODC by DFMO reduces the number and size of colon adenomas and carcinomas. Elevated levels of ODC have also been reported in transitional cell carcinoma of the bladder and the use of DFMO as a treatment for bladder cancer patients has been reported. One of the unfortunate side effects of DFMO treatment is disruption of auditory function. Accordingly, the synergistic effect provided in the present invention is useful for treatment of cancer using DFMO with low concentrations, and therefore, with less side effect.

Toxicity of DFMO can be greatly reduced by using the D-enantiomer of DFMO, or mixtures of D- and L-isomers which are enriched for the D-isomer content such that the D-isomer comprises at least 60%, and preferably more than 90% by weight of the isomeric mixture. D-DFMO, while still an inhibitor of ODC, has lower toxicity, including ototoxicity, in animal models. In a study on guinea pigs, the enantiomers of DFMO did not show significant toxicity. The D-form of DFMO was found to have no significant effects on either the compound action potential or cochlear microphonic. An evaluation of auditory function found that the L-form of DFMO produced significant disruption of normal cochlear potentials.

The use of D-DFMO or enriched D-isomer mixtures can overcome many of the problems associated with the use of racemic (50/50) D,L-DFMO. D-DFMO or enriched D-DFMO isomer mixtures may be administered at a dosage higher than a racemic mixture, due to lower anticipated toxicity associated with the D enantiomer. In three separate studies using concentrations from 0.6 μM to 80 μM D-, L-, and D,L-DFMO, the effective concentration level of each which inhibits 50% of the ODC activity (Kᵢ) can be determined. Both enantiomers, as well as the racemic mixture, were inhibitory. The Kᵢ of D-DFMO can be four fold lower than the L-form and 3 fold lower than the mixture.

DFMO and its use in the treatment of benign prostatic hypertrophy are described in two patents, U.S. Pat. Nos. 4,413,141 and 4,330,559. U.S. Pat. No. 4,413,141 describes DFMO as being an inhibitor of ODC, both in vitro and in vivo. Administration of DFMO reportedly causes a decrease in putrescine and spermidine concentrations in cells in which these polyamines are normally actively produced. Additionally, DFMO has been shown to be capable of slowing neoplastic cell proliferation when tested in standard tumor models. U.S. Pat. No. 4,330,559 describes the use of DFMO and DFMO derivatives for the treatment of benign prostatic hypertrophy. Benign prostatic hypertrophy, like many disease states characterized by rapid cell proliferation, is accompanied by abnormal elevation of polyamine concentrations. The treatment described within this reference can be administered to a patient either orally, or parenterally.

In addition, the growth of six human tumors (three mammary carcinomas, a malignant melanoma, a bladder carcinoma, and an endocervical carcinoma) can be significantly decreased after DFMO treatment compared to growth in control mice. The effect of DFMO can also be observed in xenographs of human breast and colon carcinoma cells inoculated into nude mice.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention. The following examples are intended to illustrate but not limit the invention.

### Example 1

### Treatment and Measurement of Intracellular Polyamines

DFMO (2- or α-difluoromethylornithine) can be obtained from Merrell Dow Pharmaceutical Inc. (Cincinnati, Ohio). Polyamine analogues are provided by Progen Pharmaceuticals Ltd. (Queensland, Australia). Stock solutions of each compound are diluted with medium to the desired concentrations for specific experiments. HCT116 colorectal carcinoma cells are maintained in McCoy's 5A medium supplemented with 9% FBS (Atlanta Biologicals, Lawrenceville, Ga.) and 1% penicillin/streptomycin (Mediatech, Manassas, Va.), and grown at 37° C. in 5% CO₂ atmosphere. For experiments, HCT116 cells are first treated for 24 hours with 5 mM DFMO followed by another 24 hours treatment of replenished 5 mM DFMO and polyamine analogues in the indicated doses alone or simultaneously. Intracellular polyamine concentrations are determined by high pressure liquid chromatography.

Western Blotting: Nuclear fractions are prepared using NE-PER Nuclear and Cytoplasmic Extraction reagents (Pierce, Rockford, Ill.). Equal amounts (50 μg/lane) of nuclear protein are fractionated on SDS-PAGE gels and transferred onto PVDF membranes. Primary antibody against H3K4me2 is from Millipore (Billerica, Mass.). The PCNA polyclonal antibody used for loading control is purchased from Calbiochem (Gibbstown, N.J.). Dye-conjugated secondary antibodies are used and relative protein expression levels are determined by quantitative Western analysis using the Odyssey infrared detection system and software (LI-COR Biosciences, Lincoln, Nebr.).

### Example 2

### RNA Isolation and qPCR Analysis

RNA Isolation and qPCR: RNA is extracted using TRIzol reagents (Invitrogen, Carlsbad, Calif.). First-strand cDNA is synthesized using M-MLV reverse transcriptase with an oligo(dT) primer (Invitrogen). qPCR is performed in a MyiQ single color real-time PCR machine (Bio-Rad, Hercules, Calif.) with GAPDH as an internal control. The SFRP2 primers used are: sense, 5′ AAG CCT GCA AAA ATA AAA ATG ATG (SEQ ID NO: 1); antisense, 5′ TGT AAA TGG TCT TGC TCT TGG TCT (SEQ ID NO: 2) (annealing at 53° C.).

Chromatin Immunoprecipitation (ChIP): CHIP analysis is performed using EZ-chip kit (Millipore) according to the manufacturer's instruction. In brief, cells are exposed to 1% formaldehyde to cross-link proteins, and two million cells are used for each CHIP assay. Antibody against H3 can be obtained from Abeam (Cambridge, Mass.) and antibody against H3K4me2 can be obtained from Millipore. Quantitative ChIP is performed using qPCR on the MyiQ single color real-time PCR machine. The PCR primer sets used for amplification of precipitated SFRP2 promoter fragments are as follows: sense, 5′ CTC CCT CCC AGC CTG CCC ATC TT (SEQ ID NO: 3); antisense, 5′ ACT GCC CAC CAT TTC CCC GTT TTG (SEQ ID NO: 4) (annealing at 61° C.).

### Example 3

### Combination Treatments Using Oligoamines with DFMO

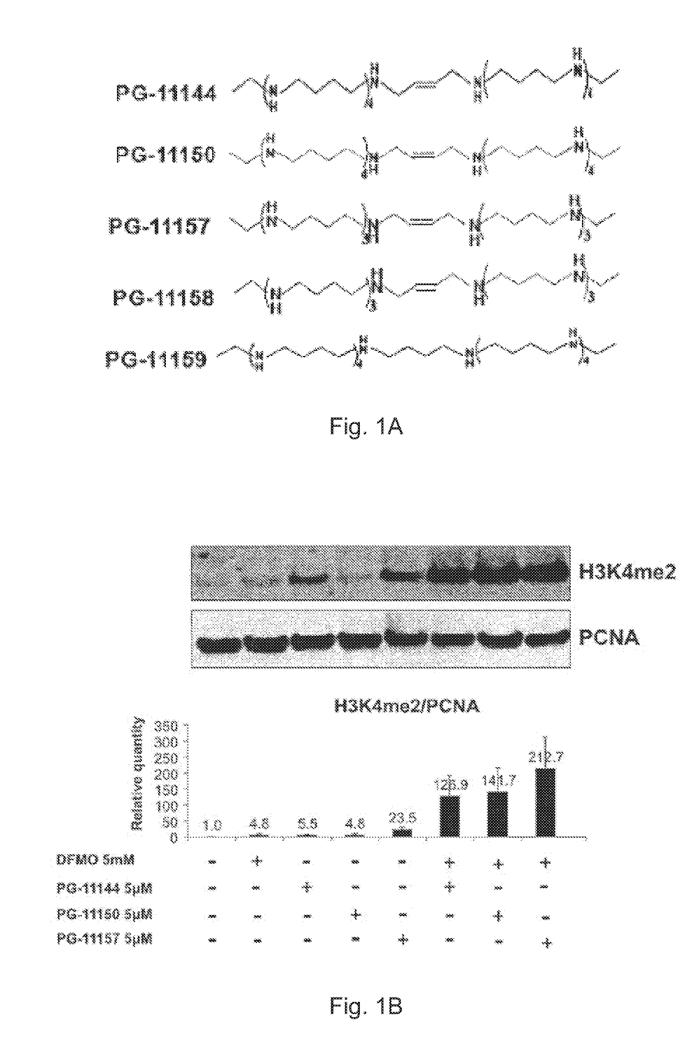

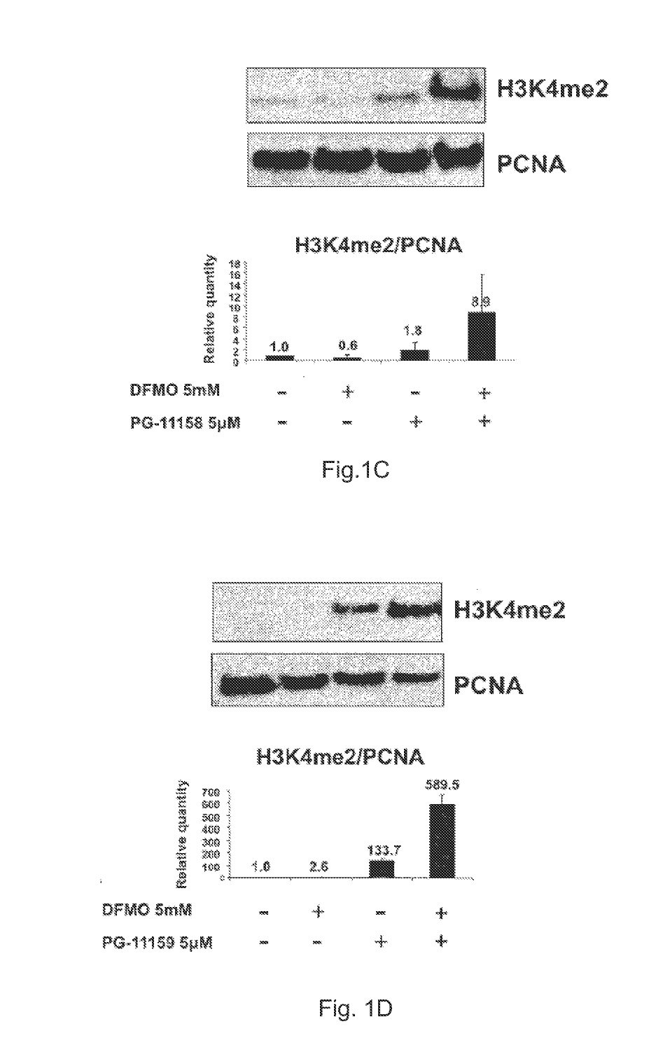

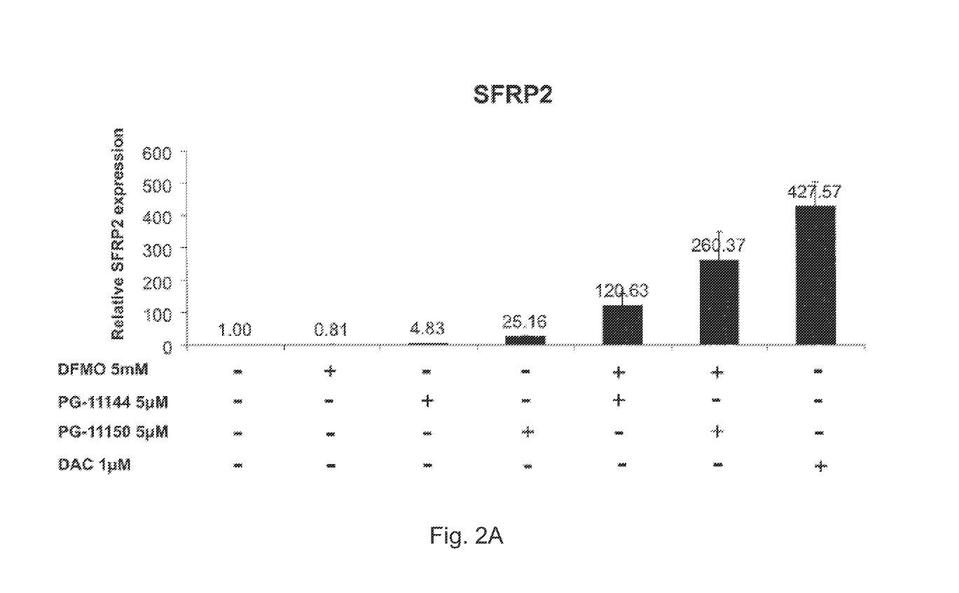

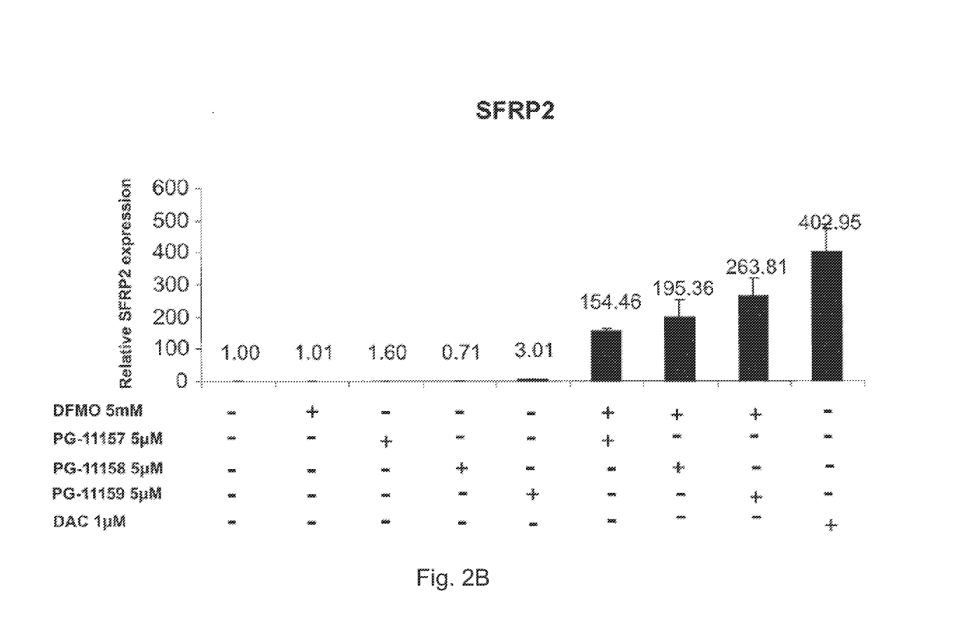

The combined treatment of DFMO with oligoamines increases global levels of H3K4me2. The present invention provides that certain specific oligoamines are effective inhibitors of LSD1. The present invention also provides that by pre-treating tumor cells with DFMO, the resulting decrease in intracellular polyamines can lead to increased effectiveness of the oligoamines in inhibiting LSD1 thus resulting in increased levels of H3K4me2, a substrate of the transcriptionally repressive LSD1 enzyme. The present invention further provides that even though each of the oligoamines are effective alone in increasing global H3K4me2 levels the combination of 5 mM DFMO with 5 μM of any of the oligoamines resulted in a massive increase in global H3K4me2 levels, indicating synergy when cells are pre-treated with DFMO (FIG. 1). These results suggest that the intracellular decrease of polyamines by DFMO pre-treatment results in greater inhibition of LSD1 by the oligoamines.

The combination of DFMO plus oligoamines results in synergistic re-expression of an aberrantly silenced gene. LSD1 is a part of transcriptional repressor complexes and its activity is associated with transcriptional repression of aberrantly silenced genes in cancer. Since there is functional synergistic inhibition of LSD1 when oligoamines and DFMO are combined, the present invention provides that global increases in H3K4me2 can be mirrored by increases in expression of previously silenced genes. Therefore, the present invention provides that the expression of the Wnt-signaling antagonist, secreted frizzle-related protein 2 (SFRP2), a gene that is frequently silenced in colon cancers as represented by the HCT116 colorectal cancer cell line, can be significantly induced by the combination of DFMO and oligoamines. The results indicate that pre-treatment of HCT116 cells with 5 mM DFMO, followed by 5 μM of the selected oligoamines all resulted in the synergistic re-expression of the previously silenced gene.

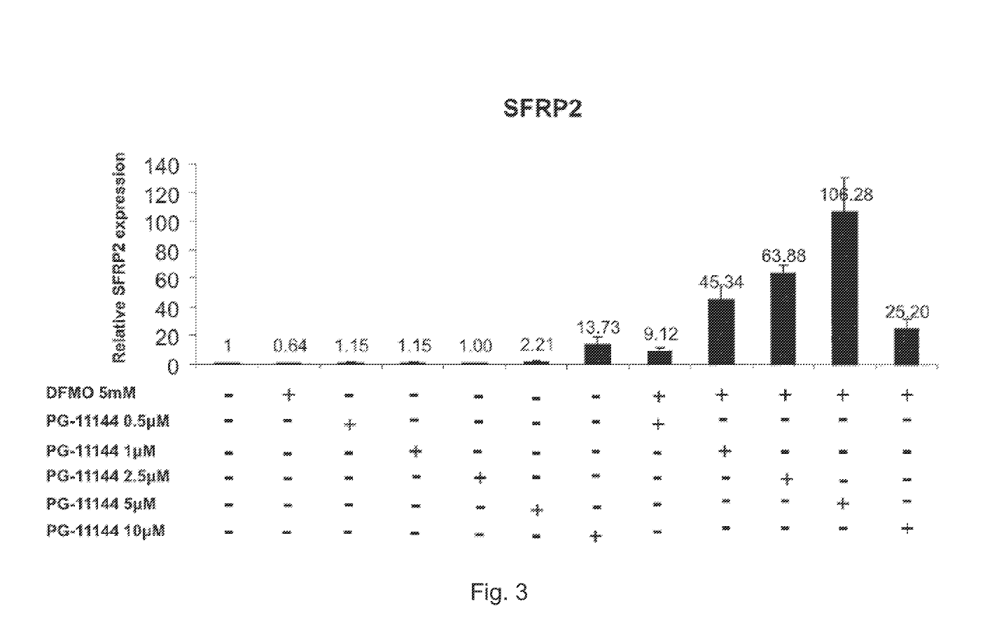

The combination of DFMO and the oligoamine PG-11144 displays dose-dependency with respect to increased PG-11144 concentration and synergistic re-expression of SFRP2. The present invention provides that PG-11144 is effective in treating established tumors in a nude mouse model and its antitumor activity is linked with functional inhibition of LSD1. Therefore, the present invention also provides the dose dependency of SFRP2 gene re-expression with increasing concentrations of PG-11144 with 5 mM DFMO. The present invention provides that 5 μM PG-11144 results in the greatest synergy with DFMO (FIG. 3). Higher concentrations actually resulted in less SFRP2 expression, presumably a result of increased cytotoxicity.

The combination of DFMO with PG-11144 results in increased H3K4me2 in the promoter region of the SFRP2 gene. The present invention provides that the re-expression of SFRP2 are directly linked to LSD1 inhibition and changes to chromatin that favors transcription, and the level of H3K4me2 in the promoter of SFRP2 can increase when cells are treated with the combination. ChIP analysis of the promoter region of SFRP2 clearly demonstrates a significant increase in the transcriptional activating mark, H3K4me2 (FIG. 4). The present invention provides that the 2.5 μM concentration of PG-11144 alone does not lead to increased SFRP2 expression (FIG. 3) or increases in the promoter H3K4me2 levels (FIG. 4). The increase of SFRP2 and H3K4 methylation only occurs when PG-11144 is combined with DFMO.

### Example 4

### Effects of Disclosed Combinations on Epigenetic Silencing of Gene Expression

Epigenetic silencing of gene expression plays a key role in the etiology and progression of cancer. Strategies to reverse aberrant gene silencing have been demonstrated to be efficacious in specific cancers and further clinical trials are ongoing to evaluate drugs targeting epigenetic regulation of gene expression. Targeting epigenetic changes is an attractive strategy as these changes, unlike gene loss or mutations, are reversible. To date, most drugs studied to alter epigenetic gene regulation have targeted either the DNA methyltransferases (DNMTs) or the histone deacetylases (HDACs). However, other significant targets exist. With the discovery of the transcriptionally repressive lysine specific demethylase, LSD1, the present invention provides that inhibition of this enzyme can lead to the re-expression of some aberrantly silenced genes. As the FAD-dependent amine oxidase LSD1 is structurally and mechanistically homologous to the polyamine oxidases, the present invention provides that certain specific polyamine analogues can effectively inhibit LSD1 activity and lead to gene re-expression. The present invention also provides that specific polyamine analogues and/or combinations of the invention can inhibit LSD1, increase promoter bound levels of H3K4me2, and lead to re-expression of previously silenced genes.

The present invention further provides that pretreatment of human colon cancer cells with the ornithine decarboxylase inhibitor, DFMO, results in intracellular polyamine depletion, and acts in a synergistic manner with the oligoamine analogues, with respect to inhibition of LSD1, increased both local and global H3K4me2 levels, and re-expression of an aberrantly silenced gene. The present invention provides that the reduction of cellular polyamine pools is necessary for the observed effects.

The natural polyamines are known to play a role in chromatin structure and there cationic nature at physiological pH makes them important counter ions to the phosphate backbone of DNA. The present invention provides that the reduction of polyamines alters chromatin conformation and/or makes the target LSD1 more accessible for the oligoamine inhibitors. It should be noted, however, that there are no data to indicate that the natural polyamines are inhibitors of, or substrates for LSD1.

The combination of DFMO and the oligoamines may have particular significance in colon cancer. Recent chemoprevention studies by Gerner, Meyskens, and colleagues demonstrate that DFMO is a promising agent for the prevention of sporadic colorectal adenomas. These studies combined with the present invention showing that PG-11144 alone, is effective in shrinking established HCT116 human colon tumors in nude mice.

Increased intracellular polyamines have been implicated in alterations in the histone acetyltransferases and HDACs, potentially resulting in epigenetic changes that lead to the initiation and progression of tumors in a transgenic mouse model where ODC, the target of DFMO, is over expressed. Another recent report indicates that polyamine depletion by DFMO can induce differentiation in cardiac myocytes through epigenetic mechanisms. Taken together, the present invention provides that inhibition of polyamine synthesis by DFMO can reduce or block, some epigenetic changes necessary for cancer. Thus the effective combination of LSD1 inhibitors with DFMO may be a result of multiple beneficial mechanisms.

In summary, it has been demonstrated that the novel combination of the LSD1-inhibiting oligoamines with the ODC inhibitor, DFMO, results in increased expression of an important Wnt-signaling antagonist that is silenced in many colon cancers. Importantly, this combination results in a synergistic response in treated cells at the level of global and local levels of the LSD1 target, H3K4me2 and expression of the SFRP2 gene. This combination represents promising new and entirely unique strategy for the targeting of epigenetically silenced genes in the treatment of cancer.

### Example 5

### Effects of Disclosed Combinations on Natural Polyamine Metabolism

DFMO treatments have been shown to alter natural polyamine metabolism in cells. The present invention provides that DFMO treatments can enhance transports of polyamines (oligoamines) of the invention into cells. The present invention further provides that such enhancement of polyamine transport involved a mechanism distinct from DFMO's effects on natural polyamine metabolism.

Table 1 shows effects of the combination treatment of DFMO and the oligoamine PG-11144 on polyamine pools in HCT116 cells. A mixture of both D-enantiomer of DFMO and L-enantiomer of DFMO is used to generate data in Table 1.

The data in Table 1 indicate that the observed synergy between DFMO and the oligoamine PG-11144 is not simply due the effect of the combination on natural polyamine metabolism. In fact the combination of DFMO and PG-11144 actually leads to a less dramatic effect on the decrease in polyamine pools as compared to DFMO treatment allow. The present invention provides that the unexpected synergy between DFMO and PG-11144 on histone modification and gene re-expression are surprising in view of the observed changes in polyamine homeostasis. Thus, the synergistic effect of disclosed combinations is not simply an accumulated result from either DFMO or polyamines/oligoamines alone, but involves a different mechanism from natural polyamine metabolism.

TABLE 1
Effects of the combination treatment
of DFMO and the oligoamine PG-11144

| | Polyamines (nmol/mg Protein)¹ | | |
|---|---|---|---|
| Treatment² | Putresine | Spermidine | Spermine |
| Control | 3.5 | 14.6 | 18.4 |
| PG-11144 0.5 μM | 4.7 | 9.1 | 11.2 |
| PG-11144 1 μM | 4.7 | 8.4 | 10.1 |
| PG-11144 2.5 μM | 3.8 | 5.6 | 6.2 |
| PG-11144 5 μM | 0.0 | 4.2 | 4.9 |
| PG-11144 10 μM | 0.0 | 4.1 | 6.4 |
| DFMO 5 mM | 0.0 | 0.0 | 13.9 |
| DFMO 5 mM + PG-11144 0.5 μM | 0.0 | 2.0 | 11.2 |
| DFMO 5 mM + PG-11144 1 μM | 0.0 | 2.9 | 10.9 |
| DFMO 5 mM + PG-11144 2.5 μM | 0.0 | 2.7 | 10.2 |
| DFMO 5 mM + PG-11144 5 μM | 0.0 | 2.3 | 13.3 |
| DFMO 5 mM + PG-11144 10 μM | 0.0 | 1.5 | 14.4 |

¹Values represent the mean of 2 determinations from a representative experiment.
²Where indicated, HCT 116 cells are pretreated with DFMO for 24 hours prior to an additional 24-hour treatment with the indicated concentration of PG-11144.

Although the invention has been described with reference to the above examples, it will be understood that modifications and variations are encompassed within the spirit and scope of the invention. Accordingly, the invention is limited only by the following claims.

1. A composition comprising
(a) a therapeutically effective amount of at least one inhibitor of a histone demethylase enzyme; and
(b) a therapeutically effective amount of at least one inhibitor of ornithine decarboxylase (ODC).

2. The composition of claim 1, wherein the histone demethylase enzyme comprises lysine-specific demethylase 1 (LSD1).

3. The composition of claim 2, wherein the inhibitor of LSD1 comprises a polyamine.

4. The compositions of claim 1, with the proviso that the inhibitor of a histone demethylase enzyme does not comprise a natural polyamine.

5. The composition of claim 1, wherein the histone demethylase enzyme comprises Jumonjii domain-containing (JmjC) histone demethylase.

6. The composition of claim 5, wherein the JmjC histone demethylase is PHF8 or KIAA1718.

7. The composition of claim 1, wherein the inhibitor of ODC comprises 2-difluoromethylornithine (DFMO or alpha-difluoromethylornithine).

8. The composition of claim 7, wherein the inhibitor of ODC comprises enriched D-enantiomer of DFMO.

9. The composition of claim 3, wherein the polyamine comprises a compound of formula (I): 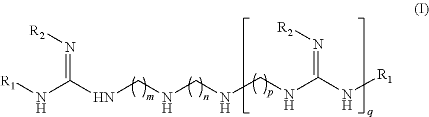
or a pharmaceutically acceptable salt thereof, wherein:
n is an integer from 1 to 12;
m and p are each independently an integer from 1 to 5;
q is 0 or 1;
each R₁ is independently selected from the group consisting of:
C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, and C₇-C₂₄ substituted or unsubstituted heteroaralkyl and;
each R₂ is independently selected from hydrogen or a C₁-C₈ substituted or unsubstituted alkyl.

10. The composition of claim 3, wherein the comprises an oligoamine of formula (X): 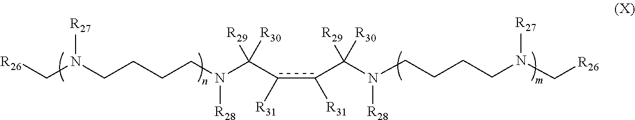
or a pharmaceutically acceptable salt thereof, wherein:
n and m are each independently an integer from 1 to 12;
each R₂₆, R₂₇, R₂₈, R₂₉, R₃₀, and R₃₁ is independently selected from hydrogen, a C₁-C₈ substituted or unsubstituted alkyl, a C₆-C₂₀ substituted or unsubstituted aryl, and an amine; and [symbol] is a single bond or double bond.

11. The composition of claim 3, wherein the compound is selected from 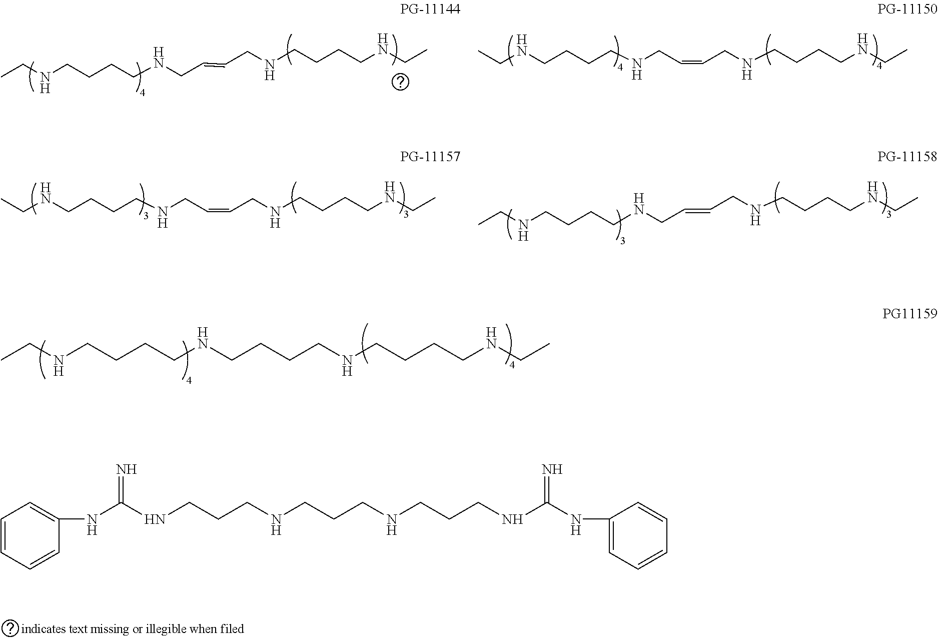
and a combination thereof.

12. A method for treatment of cancer in a subject comprising:
administering to the subject a therapeutically effective amount of at least one inhibitor of a histone demethylase enzyme in combination with a therapeutically effective amount of at least one inhibitor of ornithine decarboxylase (ODC).

13. The method of claim 12, wherein the inhibitor of ODC comprises 2-difluoromethylornithine (DFMO or alpha-difluoromethylornithine) and the inhibitor of a histone demethylase enzyme comprises a polyamine.

14. The method of claim 13, with the proviso that the inhibitor of a histone demethylase enzyme does not comprise a natural polyamine.

15. The method of claim 13, wherein the polyamine comprises a compound of formula (I) or formula (X): 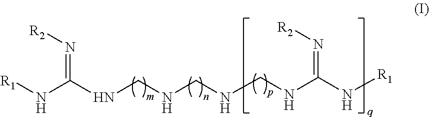
or a pharmaceutically acceptable salt thereof wherein:
n is an integer from 1 to 12;
m and p are each independently an integer from 1 to 5;
q is 0 or 1;
each R₁ is independently selected from the group consisting of:
C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, and C₇-C₂₄ substituted or unsubstituted heteroaralkyl and;
each R₂ is independently selected from hydrogen or a C₁-C₈ substituted or unsubstituted alkyl; or 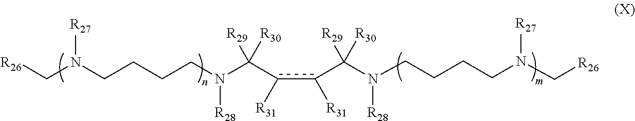
or a pharmaceutically acceptable salt thereof, wherein:
n and m are each independently an integer from 1 to 12;
each R₂₆, R₂₇, R₂₈, R₂₉, R₃₀, and R₃₁ is independently selected from hydrogen, a C₁-C₈ substituted or unsubstituted alkyl, a C₆-C₂₀ substituted or unsubstituted aryl, and an amine; and [symbol] is a single bond or double bond.

16. The method of claim 14, wherein the compound is selected from 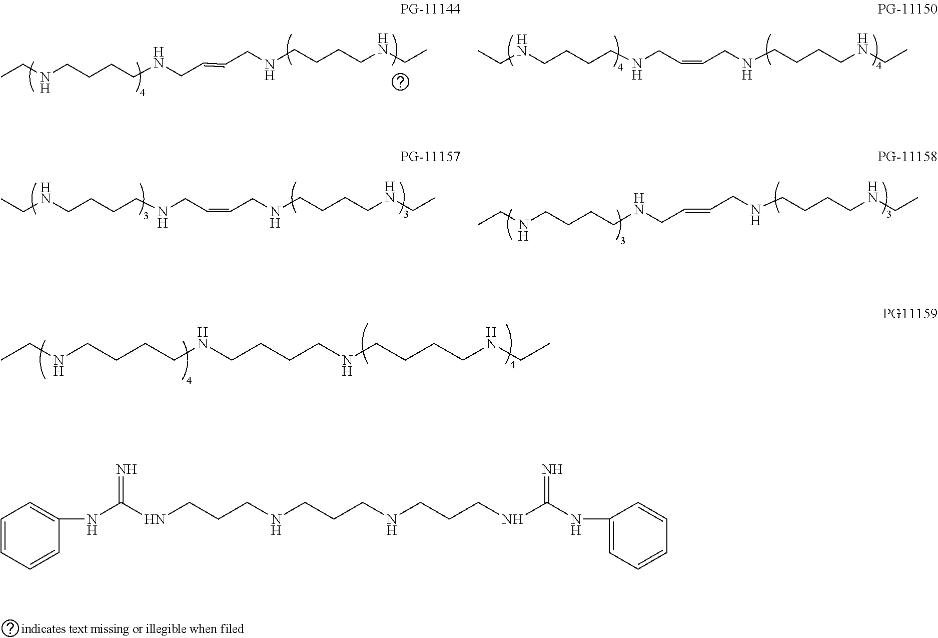
and a combination thereof.

17. A method of altering DNA methylation in a cell comprising
administering the cell with at least one inhibitor of a histone demethylase enzyme in combination with at least one inhibitor of ornithine decarboxylase (ODC).

18. The method of claim 17, wherein the inhibitor of ODC comprises 2-difluoromethylornithine (DFMO or alpha-difluoromethylornithine) and the inhibitor of a histone demethylase enzyme comprises a polyamine.

19. The method of claim 18, with the proviso that the inhibitor of a histone demethylase enzyme does not comprise a natural polyamine.

20. The method of claim 17, wherein the polyamine comprises a compound of formula (I) or formula (X): 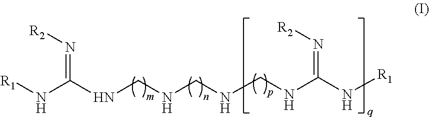
or a pharmaceutically acceptable salt thereof, wherein:
n is an integer from 1 to 12;
m and p are each independently an integer from 1 to 5;
q is 0 or 1;
each R₁ is independently selected from the group consisting of:
C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, and C₇-C₂₄ substituted or unsubstituted heteroaralkyl and;
each R₂ is independently selected from hydrogen or a C₁-C₈ substituted or unsubstituted alkyl; or 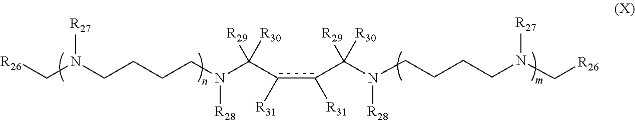
or a pharmaceutically acceptable salt thereof, wherein:
n and m are independently an integer from 1 to 12;
each R₂₆, R₂₇, R₂₈, R₂₉, R₃₀, and R₃₁ is independently selected from hydrogen, a C₁-C₈ substituted or unsubstituted alkyl, a C₆-C₂₀ substituted or unsubstituted aryl, and an amine; and [symbol] is a single bond or double bond.

21. The method of claim 20, wherein the compound is selected from 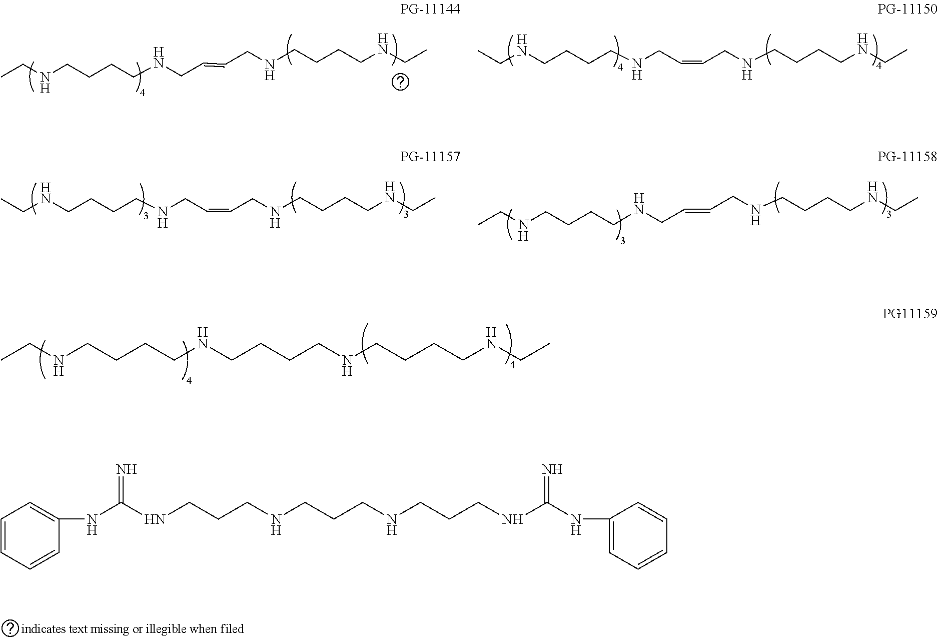
and a combination thereof.

22. A method for enhancing inhibition of a histone demethylase enzyme in a cell comprising:
administering the cell with at least one inhibitor of ornithine decarboxylase (ODC); and
administering the cell with at least one inhibitor of a histone demethylase enzyme.

23. The method of claim 22, wherein the inhibitor of ODC comprises 2-difluoromethylornithine (DFMO or alpha-difluoromethylornithine) and the inhibitor of a histone demethylase enzyme comprises a polyamine.

24. The method of claim 23, with the proviso that the inhibitor of a histone demethylase enzyme does not comprise a natural polyamine.

25. The method of claim 22, wherein the step (a) comprises a pretreatment period from about 2 hours to about 48 hours.

26. The method of claim 23, wherein the polyamine comprises a compound of formula (I) or formula (X): 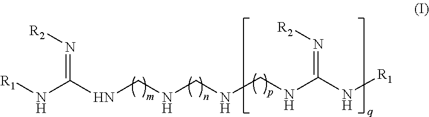
or a pharmaceutically acceptable salt thereof wherein:
n is an integer from 1 to 12;
m and p are each independently an integer from 1 to 5;
q is 0 or 1;
each R₁ is independently selected from the group consisting of:
C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, and C₇-C₂₄ substituted or unsubstituted heteroaralkyl and;
each R₂ is independently selected from hydrogen or a C₁-C₈ substituted or unsubstituted alkyl; or 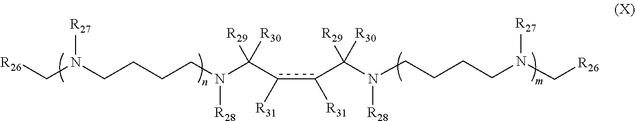
or a pharmaceutically acceptable salt thereof, wherein:
n and m are independently an integer from 1 to 12;
each R₂₆, R₂₇, R₂₈, R₂₉, R₃₀, and R₃₁ is independently selected from hydrogen, a C₁-C₈ substituted or unsubstituted alkyl, a C₆-C₂₀ substituted or unsubstituted aryl, and an amine; and [symbol] is a single bond or double bond.

27. The method of claim 26, wherein the compound is selected from 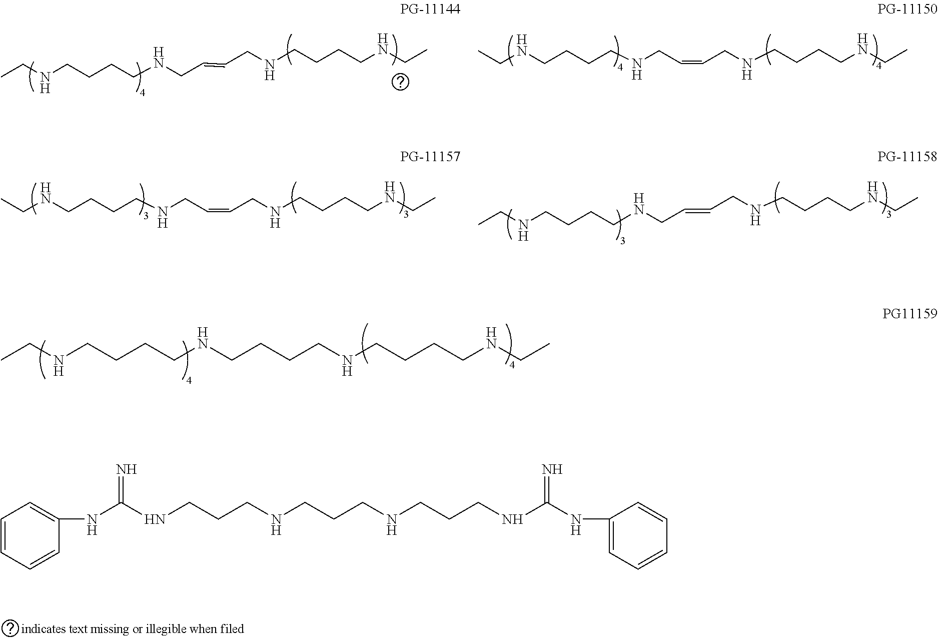
and a combination thereof.

28. The method of claim 12, wherein the subject is human.

29. The method of claim 17, wherein the cell is a cancer cell.

30. The method of claim 12, wherein the cancer is selected from the group consisting of bladder, brain, breast, colon, esophagus, kidney, liver, lung, mouth, ovary, pancreas, prostate, skin, stomach, hematopoietic system and uterus.

31. The method of claim 30, wherein the hematopoietic cancers comprise at least one of acute myeloid leukemia, mesothelioma, cutaneous T-cell lymphoma (CTCL), multiple myeloma and myelodysplastic syndrome (refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) or combinations thereof.

32. The use of at least one inhibitor of a histone demethylase enzyme in combination with at least one inhibitor of ornithine decarbosylase (ODC) in the manufacture of a medicament for treating cancer in a subject.

33. A combination of at least one inhibitor of a histone demethylase enzyme and at least one inhibitor of ornithine decarbosylase (ODC) for use in a method of treating cancer in a subject.

34. The method of claim 22, wherein the cell is a cancer cell.


SEQUENCE LISTING (4 sequences)

SEQ ID NO: 1 - DNA, 24 bp, Artificial Sequence
  Primer
aagcctgcaaaaataaaaatgatg

SEQ ID NO: 2 - DNA, 24 bp, Artificial Sequence
  Primer
tgtaaatggtcttgctcttggtct

SEQ ID NO: 3 - DNA, 23 bp, Artificial Sequence
  Primer
ctccctcccagcctgcccatctt

SEQ ID NO: 4 - DNA, 24 bp, Artificial Sequence
  Primer
actgcccaccatttccccgttttg